(19) United States
(12) Patent Application Publication
(10) Pub. No.: US 2009/0215627 A1
(43) Pub. Date: Aug. 27, 2009

(54) Crystal Structure of Biotin Carboxylase (Bc) Domain of Acetyl-Coenzyme a Carboxylase and Methods of Use Thereof

(76) Inventors: Yang Shen, New York, NY (US); Sandra L. Volrath, Durham, NC (US); Stephanie C. Weatherly, Durham, NC (US); Tedd D. Elich, Durham, NC (US); Richard Anderson, Palo Alto, CA (US); Liang Tong, Scarsdale, NY (US)

Correspondence Address: MYERS BIGEL SIBLEY & SAJOVEC, PO BOX 37428, RALEIGH, NC, 27627, US

(21) Appl. No.: 11/573,217

(22) PCT Filed: Aug. 3, 2005
(86) PCT No.: PCT/US05/27440

§ 371 Date: Sep. 17, 2007

Related U.S. Application Data
(60) Provisional application No. 60/599,831, filed on Aug. 6, 2004.
(60) Provisional application No. 60/637,068, filed on Dec. 17, 2004.

(51) Int. Cl. A01N 43/60; C12N 9/00; A61K 31/5513; A01P 13/00; A61P 3/10; G06G 7/48; C07D 243/08

(52) U.S. Cl. 504/218; 435/183; 540/575; 514/218; 703/11

(57) ABSTRACT

A crystal comprising a biotin carboxylase domain of acetyl-CoA carboxylase is described, along with a computer-based method for identifying compounds that modulates activity of acetyl-CoA carboxylase, a computer-based method for rationally designing a compound that modulates activity of acetyl-CoA carboxylase, along with compounds produced by such methods, as well as compositions and methods of use thereof.

### RELATED APPLICATIONS

This application claims the benefit of U.S. Provisional Patent Applications Ser. No. 60/637,068, filed Dec. 17, 2004 and Ser. No. 60/599,831, filed Aug. 6, 2004, the disclosures of both of which are incorporated by reference herein in their entirety.

This invention was made with Government support under grant nos. DK67238 and DK068962 from the National Institutes of Health. The Government has certain rights to this invention.

### BACKGROUND OF THE INVENTION

Acetyl-coenzyme A carboxylases (ACCs) have crucial roles in the metabolism of fatty acids, and therefore are important targets for drug development against obesity, diabetes and other diseases (Abu-Elheiga, L. et al., Science 291, 2613-2616 (2001); Alberts, A. W., and Vagelos, P. R. Acyl-CoA Carboxylases. In The Enzymes, P. D. Boyer, ed. (New York, Academic Press), pp. 37-82 (1972); Cronan Jr., J. E., and Waldrop, G. L., Prog Lipid Res 41, 407-435 (2002); Harwood Jr., H. J. et al., J Biol Chem 278, 37099-37111 (2003); Wakil, S. J. et al., Ann Rev Biochem 52, 537-579 (1983); Zhang, H. et al., Crystal structure of the carboxyltransferase domain of acetyl-coenzyme A carboxylase in complex with CP-640186. Structure. in press (2004a); Zhang, H. et al., Proc Natl Acad Sci USA 101, 5910-5915 (2004b); Zhang, H. et al., Science 299, 2064-2067 (2003)). ACCs catalyze the carboxylation of acetyl-CoA to produce malonyl-CoA. In mammals, ACC1 is present in the cytosol of liver and adipose tissues and controls the committed step in the biosynthesis of long-chain fatty acids. In comparison, ACC2 is associated with the outer membrane of mitochondria in the heart and muscle. Its malonyl-CoA product is a potent inhibitor of carnitine palmitoyltransferase I, which facilitates the transport of long-chain acyl-CoAs into the mitochondria for oxidation (McGarry, J. D. et al., Eur J Biochem 244, 1-14 (1997); Ramsay, R. R. et al., Biochim Biophys Acta 1546, 21-43 (2001);). The importance of ACCs for drug discovery is underscored by the observations that mice lacking ACC2 have elevated fatty acid oxidation, reduced body fat and body weight (Abu-Elheiga, L. et al., Proc Natl Acad Sci USA 100, 10207-10212 (2003); Lenhard, J. M. et al., Advanced Drug Delivery Reviews 54, 1199-1212 (2002)).

Eukaryotic ACCs are large, single-chain, multi-domain enzymes, with a biotin carboxylase (BC) domain, a biotin carboxyl carrier protein (BCCP) domain, and a carboxyltransferase (CT) domain, whereas these activities exist as separate subunits in the prokaryotic ACCs (FIG. 1A) (Abu-Elheiga et al., supra (2001); Lenhard et al., supra (2002); Wakil et al., supra). The BC activity catalyzes the ATP-dependent carboxylation of biotin (FIG. 1B), and the CT activity catalyzes the transfer of the activated carboxyl group to acetyl-CoA to produce malonyl-CoA. The amino acid sequences of the BC domains are highly conserved among the eukaryotes, with 63% sequence identity between those of yeast ACC and human ACC1 (FIG. 1C). In contrast, the sequence conservation between the eukaryotic and prokaryotic BC is much weaker. For example, there is only 35% amino acid identity between yeast and E. coli BC (FIG. 1C). Moreover, the yeast BC domain, with 570 residues, is ˜120 residues larger than the E. coli BC subunit (FIG. 1A).

Soraphen A was originally isolated from the culture broth of Sorangiuin cellulosum, a soil dwelling myxobacterium, for its potent antifungal activity (Gerth, K., et al., J Antibiot (Tokyo) 47, 23-31 (1994); Gerth, K. et al., J Biotech 106, 233-253 (2003)). This polyketide natural product contains an unsaturated 18-membered lactone ring, an extracyclic phenyl ring, two hydroxyl groups, three methyl groups, and three methoxy groups (Bedorf, N. et al., Liebigs Ann Chem 9, 1017-1021 (1993); Ligon, J. et al., Gene 285, 257-267 (2002)) (FIG. 2A). There is also a 6-membered ring within the macrocycle formed by a hemiketal between the C3 carbonyl and C7 hydroxyl (FIG. 2A). Soraphen A has demonstrated strong promise as a broad-spectrum fungicide against various plant pathogenic fungi (Pridzun, L., Untersuchungen zum Wirkungsmechanismus von Soraphen A, Technical University of Braunschweig (1991)). Genetic and biochemical studies show that soraphen A is a potent inhibitor of the BC domain of eukaryotic ACCs (Gerth et al., supra (1994; 2003); Pridzun, supra (1991); Pridzun, L. et al., Inhibition of fungal acetyl-CoA carboxylase: a novel target discovered with the myxobacterial compound soraphen. In Antifungal agents, G. K. Dixon, L. G. Copping, and D. W. Hollomon, eds. (Oxford, UK, BIOS Scientific Publishers Ltd.), pp. 99-109 (1995); Vahlensieck, H. F. et al., U.S. Pat. No. 5,641,666 (1997); Vahlensieck, H. F. et al., Curr Genet 25, 95-100 (1994)), with K_{d} values of about 1 nM. In comparison, the compound has no effect on bacterial BC subunits (Behrbohm, H., Acetyl-CoA Carboxylase aus Ustilago maydis. Reinigung, Charakterisierung und Intersuchungen zur Inhibierung durch Soraphen A, Technical University of Braunschweig (1996); Weatherly, S. C. et al., Biochem J 380, 105-110 (2004). However, it is not known how soraphen A achieves its activity and its specificity towards the eukaryotic ACCs.

### SUMMARY OF THE INVENTION

A first aspect of the present invention is a crystal comprising a biotin carboxylase domain of acetyl-CoA carboxylase.

A second aspect of the invention is a computer-based method for identifying compounds that modulates activity of acetyl-CoA carboxylase comprising: (a) providing at least 30 coordinates for a biotin carboxylase domain of acetyl-CoA carboxylase in a computer; (b) providing a structure of a candidate compound to said computer in computer readable form; and (c) determining whether or not said candidate compound fits into or docks with a binding cavity of said biotin carboxylase domain, wherein a candidate compound that fits or docks into said binding cavity is determined to be likely to modulate activity of acetyl-CoA carboxylase. Said compound may, for example, be a member of a compound library.

A further aspect of the invention is a computer-based method for rationally designing a compound that modulates activity of acetyl-CoA carboxylase, comprising: (a) generating a computer readable model of a binding site of a biotin carboxylase domain of acetyl-CoA carboxylase; and then (b) designing in a computer with said model a compound having a structure and a charge distribution compatible with said binding site, said compound having a functional group that interacts with said binding site to modulate acetyl-CoA carboxylase activity.

A further aspect of the invention is a computer readable medium comprising the methods described above.

A further aspect of the invention is a data structure comprising atomic coordinates for a biotin carboxylase domain of acetyl-CoA carboxylase.

A further aspect of the invention is a computer displaying a virtual model of a biotin carboxylase domain of acetyl-CoA carboxylase.

A further aspect of the invention is a storage medium containing atomic coordinates for a biotin carboxylase domain of acetyl-CoA carboxylase.

A further aspect of the invention is a compound produced by a method as described herein.

A further aspect of the invention is a method of treating a plant comprising administering a treatment-effective amount of a compound identified by a method as described herein to said plant (e.g., an amount effective to inhibit, control, or combat a fungal infection of said plant).

A further aspect of the invention is a method of treating metabolic syndrome, insulin resistance syndrome or obesity in a subject in need of such treatment, comprising administering to said subject a treatment-effective amount of a compound identified by a method as described herein.

The foregoing and other objects and aspects of the present invention are described in greater detail in the drawings herein and the specification set forth below.

### BRIEF DESCRINTION OF THE DRAWINGS

FIG. 1. The biotin carboxylase (BC) domain of acetyl coenzyme-A carboxylase (ACC). (A). Domain organization of yeast ACC (top) and the subunits of E. coli ACC (bottom). BC-biotin carboxylase; BCCP-biotin carboxyl carrier protein; CT-carboxyltransferase. (B). The reaction catalyzed by the BC activity. (C). Sequence alignment of the BC domains of yeast ACC (SEQ ID NO:10) and human ACC1(SEQ ID NO:11), and the BC subunit of E. coli ACC (SEQ ID NO:12). Residues involved in binding soraphen are highlighted in green, and in red for Ser77. Residues in the dimer interface of E. coli BC are highlighted in magenta. Residues in bacterial BC that are structurally equivalent to those in yeast BC are shown in upper case. S.S.-secondary structure.

FIG. 2. Structure of biotin carboxylase (BC) in complex with soraphen A. (A). Chemical structure of soraphen A. The numbering scheme of atoms in the macrocycle is shown. (B). Final 2Fₒ-F_{c} electron density at 1.8 Å resolution for soraphen A, contoured at 1σ. Produced with Setor (Evans, S. V., J Mol Graphics 11, 134-138 (1993)). (C). Schematic drawing of the structure of yeast BC domain in complex with soraphen A. Residues 535-538 (in the αR-αS loop) are disordered in this molecule and are shown in gray. Soraphen A is shown as a stick model in green for carbon atoms, labeled Sor. The expected position of ATP, as observed in the E. coli BC subunit (Thoden, J. B. et al., J Biol Chem 275, 16183-16190 (2000)), is shown in gray. (D). Side view of the structure of the BC:soraphen complex. The different domains are colored differently. Panels C and D produced with Ribbons (Carson, M., J Mol Graphics 5, 103-106 (1987).

FIG. 3. The binding mode of soraphen A. (A). Stereographic drawing showing the binding site for soraphen A. Produced with Ribbons (Carson, supra (1987)). (B). Schematic drawing of the interactions between soraphen A and the BC domain. (C). Molecular surface of the BC domain in the soraphen binding site. Produced with Grasp (Nicholls, A. et al., Proteins 11, 281-296 (1991)).

FIG. 4. Conformational differences in the bacterial BC subunit precludes soraphen binding. (A). Schematic drawing of the structure of E. coli BC subunit in complex with ATP (Thoden et al., supra (2000). Regions of large structural differences to the yeast BC domain are indicated with red arrows. (B). Structural comparison between yeast (in yellow) and E. coli (cyan) BC in the soraphen binding site. (C). Molecular surface of the E. coli BC in the soraphen binding site. The soraphen molecule is shown for reference, and has extensive steric clash with the bacterial BC. Panels A and B produced with Ribbons (Carson, 1987), and panel C with Grasp (Nicholls et al., supra 1991).

FIG. 5. Fluorescence-based assay for soraphen binding to the BC domain. Trp emission at 340 nm for the wild-type, K73R, and E477R mutants is plotted as a function of the soraphen concentration. The curves represent fits to a one-site binding model.

FIG. 6. Only minor structural changes in the BC domain upon soraphen binding. (A). Structural overlay of the free enzyme (in yellow) and soraphen complex (cyan) of yeast BC domain. The positions of soraphen (green) and ATP (gray) are shown for reference. (B). Structural overlay of the soraphen binding site in the free enzyme (yellow for main chain, magenta for side chain) and the soraphen complex (cyan and gray).

FIG. 7. Soraphen A may disrupt the oligomerization of the BC domain. (A). Schematic drawing of the dimer of E. coli BC subunit in complex with ATP (Thoden et al., supra 2000). The dimer axis is indicated with the magenta oval. The position of soraphen as observed in the yeast BC domain structure is shown for reference. (B). Native gel (12%) showing the electrophoretic mobility of wild-type and K73R mutant of yeast BC domain in the absence or presence of soraphen. Possible bands in the gel are marked with the arrowheads. Each lane was loaded with 20 μg of protein in 10 μl.

### DETAILED DESCRIPTION OF THE PREFERRED EMBODIMENTS

A biotin carboxylase (BC) domain of Acetyl CoA carboxylase may be produced in accordance with known techniques including but not limited to those described in T. Elich et al., PCT Application WO 2004/013159, titled Recombinant Biotin Carboxylase Domains for Identification of Acetyl CoA Carboxylase Inhibitors.

For example, the design of constructs for expression of the two human ACC BC domains can be based on homology to the U. maydis BC domain of pCS8 as shown in Table 1A below. Excluding N-terminal extensions, these BC domains are 63% identical.

TABLE 1A

| | |
|---|---|
| Ustilago | -ASPVADFIRKQGGHSVITKVLICNNGIAAVKEIRSIRKWAYETFGDERAIEFTVMATPE |
| ACC1 | VASP-AEFVTRFGGNKVIEKVLIANNGIAAVKCMRSIRRWSYEMFRNERAIRFVVMVTPE |
| ACC2 | VASP-AEFVTRFGGDRVIEKVLIANNGIAAVKCMRSIRRWAYEMFRNERAIRFVVMVTPE |
| | *** * * ** ** **** ******** **** * ** * **** * ** *** |
| Ustilago | DLKVNADYIRMADQYVEVPGGSNNNNYANVDLIVDVAERAGVHAVWAGWGHASENPRLPE |
| ACC1 | DLKANAEYIKMADHYVPVPGGPNNNNYANVELILDIAKRIPVQAVWAGWGHASENPKLPE |
| ACC2 | DLKANAEYIKMADHYVPVPGGPNNNNYANVELIVDIAKRIPVQAVWAGWGHASENPKLPE |
| | *** ** ** *** ** **** ******** ** * * * * ************* *** |
| Ustilago | SLAASKHKIIFIGPPGSAMRSLGDKISSTIVAQHADVPCMPWSGTGIKETMMSD---QGF |
| ACC1 | LLL--KNGIAFMGPPSQAMWALGDKIASSIVAQTAGIPTLPWSGSGLRVDWQENDFSKRI |
| ACC2 | LLC--KNGVAFLGPPSEAMWALGDKIASTVVAQTLQVPTLPWSGSGLTVEWTEDDLQQGK |
| | * * * *** ** ***** * *** * **** * |
| Ustilago | -LTVSDDVYQQACIHTAEEGLEKAEKIGYPVMIKASEGGGGKGIRKCTNGEEFKQLYNAV |
| ACC1 | -LNVPQELYEKGYVKDVDDGLQAAEEVGYPVMIKASEGGGGKGIRKVNNADDFPNLFRQV |
| ACC2 | RISVPEDVYDKGCVKDVDEGLEAAERIGFPLMIKASEGGGGKGIRKAESAEDFPILFRQV |
| | * * ** ** * * *************** * * * |
| Ustilago | LGEVPGSPVFVMKLAGQARHLEVQLLADQYGNAISIFGRDCSVQRRHQKIIEEAPVTIAP |
| ACC1 | QAEVPGSPIFVMRLAIQSRHLEVQILADQYGNAISLFGRDCSVQRRHQKIIEEAPATIAT |
| ACC2 | QSEIPGSPIFLMKLAQHARHLEVQILADQYGNAVSLFGRDCSIQRRHQKIVEEAPATIAP |
| | * **** * * ** ****** ******** * ****** ******* **** *** |
| Ustilago | EDARESMEKAAVRLAKLVGYVSAGTVEWLYSPESGEFAFLELNPRLQVEHPTTEMVSGVN |
| ACC1 | PAVFEHMEQCAVKLAKMVGYVSAGTVEYLYS-QDGSFYFLELNPRLQVEHPCTEMVADVN |
| ACC2 | LAIFEFMEQCAIRLAKTVGYVSAGTVEYLYS-QDGSFHFLELNPRLQVEHPCTEMIADVN |
| | * ** * *** ********** *** * * ************* *** ** |
| Ustilago | IPAAQLQVAMGIPLYSIRDIRTLYGMDPRGNEVIDFDFSSPESFKTQRKPQ-PQGHVVAC |
| ACC1 | LPAAQLQIAMGIPLYRIKDIRMMYGVSPWGDSPIDFEDSA-------HVPC-PRGHVIAA |
| ACC2 | LPAAQLQIAMGVPLHRLKDIRLLYGESPWG--------VTPISFETPSNPPLARGHVIAA |
| | ****** *** ** *** ** * * * *** * |
| Ustilago | RITAENPDTGFKPGMGALTELNFRSSTSTWGYFSVGTSGALHEYADSQFGHIFAYGADRS |
| ACC1 | RITSENPDEGFKPSSGTVQELNFRSNKNVWGYFSVAAAGGLHEFADSQFGHCFSWGENRE |
| ACC2 | RITSENPDEGFKPSSGTVQELNFRSSKNVWGYFSVAATGGLHEFADSQFGHCFSWGENRE |
| | *** **** **** * ****** ****** * *** ******* * * * |
| Ustilago | EARKQMVISLKELSIRGDFRTTVEYLIKLLETDAFESNKITTGWLDGLIQDRLTAERPPA |
| ACC1 | EAISNMVVALKELSIRGDFRTTVEYLIKLLETESFQMNRIDTGWLDRLIAEKVQAERPDT |
| ACC2 | EAISNMVVALKELSIRGDFRTTVEYLINLLETESFQNNDIDTGWLDYLIAEKVQAEKPDI |
| | ** ** ****************** **** * * * ***** ** ** * |

| | | |
|---|---|---|
| Ustilago | DLAV | (SEQ ID NO:1) |
| ACC1 | MLGV | (SEQ ID NO:2) |
| ACC2 | MLGV | (SEQ ID NO:3) |
| | * * | |

Alignment of the ustilago and human ACCase
BC domains (with N-termini)

| | |
|---|---|
| ustilagoBC | ------------------------------------------------------------ |
| ACC1 | MDE--------------------------------------------------------- |
| ACC2 | MVLLLCLSCLIFSCLTFSWLKIWGKMTDSKPITKSKSEANLIPSQEPFPASDNSGETPQR |
| Ustilago | --------------PPPDEKAV-----S-------------QFIGGNPLET--------- |
| ACC1 | --------------PSPLAQPLELNQHS-------------RFIIGSVSEDNSEDEISNL |
| ACC2 | NGEGHTLPKTPSQAEPASHKGP-----KDAGRRRNSLPPSHQKPPRNPLSS--------- |
| Ustilago | -------------APAS------------------------------------------- |
| ACC1 | VKLDLLEEKEGSLSPASVGSDTLSDLGISSLQDGLALHIRSSMSGLHLVKQGRDRKKIDS |
| ACC2 | -------------SDAA------------------------------------------- |
| | * |
| Ustilago | -------PV--------------------------------------------------- |
| ACC1 | QRDFTVASP--------------------------------------------------- |
| ACC2 | -------PSPELQANGTGTQGLEATDTNGLSSSARPQGQQAGSPSKEDKKQANIKRQLMT |
| ustilagoBC | ------------------------------------------------------------ |
| ACC1 | ------------------------------------------------------------ |
| ACC2 | NFILGSFDDYSSDEDSVAGSSRESTRKGSRASLGALSLEAYLTTGEAETRVPTMRPSMSG |
| ustilagoBC | ------------------------ADFIRKQGGHSVITKVLICNNGIAAVKEIRSIRKWA |
| ACC1 | ------------------------AEFVTRFGGNKVIEKVLIANNGIAAVKCMRSIRRWS |
| ACC2 | LHLVKRGREHKKLDLHRDFTVASPAEFVTRFGGDRVIEKVLIANNGIAAVKCMRSIRRWA |
| | * * ** ** **** ******** **** * |
| ustilagoBC | YETFGDERAIEFTVMATPEDLKVNADYIRMADQYVEVPGGSNNNNYANVDLIVDVAERAG |
| ACC1 | YEMFRNERAIRFVVMVTPEDLKANAEYIKMADHYVPVPGGPNNNNYANVELILDIAKRIP |
| ACC2 | YEMFRNERAIRFVVMVTPEDLKANAEYIKMADHYVPVPGGPNNNNYANVELIVDIAKRIP |
| | ** * **** * ** ****** ** ** *** ** **** ******** ** * * * |
| Ustilago | VHAVWAGWGHASENPRLPESLAASKHKIIFIGPPGSAMRSLGDKISSTIVAQHADVPCMP |
| ACC1 | VQAVWAGWGHASENPKLPELLL--KNGIAFMGPPSQAMWALGDKIASSIVAQTAGIPTLP |
| ACC2 | VQAVWAGWGHASENPKLPELLC--KNGVAFLGPPSEAMWALGDKIASTVVAQTLQVPTLP |
| | ************* *** * * * *** ** ***** * *** * * |
| Ustilago | WSGTGIKETMMSD---QGF-LTVSDDVYQQACIHTAEEGLEKAEKIGYPVMIKASEGGGG |
| ACC1 | WSGSGLRVDWQENDFSKRI-LNVPQELYEKGYVKDVDDGLQAAEEVGYPVMIKASEGGGG |
| ACC2 | WSGSGLTVEWTEDDLQQGKRISVPEDVYDKGCVKDVDEGLEAAERIGFPLMIKASEGGGG |
| | *** * * * ** ** * * ********** |
| Ustilago | KGIRKCTNGEEFKQLYNAVLGEVPGSPVFVMKLAGQARHLEVQLLADQYGNAISIFGRDC |
| ACC1 | KGIRKVNNADDFPNLFRQVQAEVPGSPIFVMRLAKQSRHLEVQILADQYGNAISLFGRDC |
| ACC2 | KGIRKAESAEDFPILFRQVQSEIPGSPIFLMKLAQHARHLEVQILADQYGNAVSLFGRDC |
| | ***** * * * * **** * * ** ****** ******** * ***** |
| Ustilago | SVQRRHQKIIEEAPVTIAPEDARESMEKAAVRLAKLVGYVSAGTVEWLYSPESGEFAFLE |
| ACC1 | SVQRRHQKIIEEAPATIATPAVFEHMEQCAVKLAKMVGYVSAGTVEYLYS-QDGSFYFLE |
| ACC2 | SIQRRHQKIVEEAPATIAPLAIFEFMEQCAIRLAKTVGYVSAGTVEYLYS-QDGSFHFLE |
| | * ******* **** *** * ** * *** ********** *** * * *** |
| Ustilago | LNPRLQVEHPTTEMVSGVNIPAAQLQVAMGIPLYSIRDIRTLYGMDPRGNEVIDFDFSSP |
| ACC1 | LNPRLQVEHPCTEMVADVNLPAAQLQIAMGIPLYRIKDIRMMYGVSPWGDSPIDFEDSA- |
| ACC2 | LNPRLQVEHPCTEMIADVNLPAAQLQIAMGVPLHRLKDIRLLYGESPWG--------VTP |
| | ********** *** ** ****** *** ** *** ** * * |
| Ustilago | ESFKTQRKPQ-PQGHVVACRITAENPDTGFKPGMGALTELNFRSSTSTWGYFSVGTSGAL |
| ACC1 | ------HVPC-PRGHVIAARITSENPDEGFKPSSGTVQELNFRSNKNVWGYFSVAAAGGL |
| ACC2 | ISFETPSNPPLARGHVIAARITSENPDEGFKPSSGTVQELNFRSSKNVWGYFSVAATGGL |
| | * *** * *** **** **** * ****** ****** * * |
| Ustilago | HEYADSQFGHIFAYGADRSEARKQMVISLKELSIRGDFRTTVEYLIKLLETDAFESNKIT |
| ACC1 | HEFADSQFGHCFS˜GENREEAISNMVVALKELSIRGDFRTTVEYLIKLLETESFQMNRID |
| ACC2 | HEFADSQFGHCFSWGENREEAISNMVVALKELSIRGDFRTTVEYLINLLETESFQNNDID |
| | ** ******* * * * ** ** ****************** **** * * * |

| | | |
|---|---|---|
| Ustilago | TGWLDGLIQDRLTAERPPADLAV | (SEQ ID NO:4) |
| ACC1 | TGWLDRLIAEKVQAERPDTMLGV | (SEQ ID NO:5) |
| ACC2 | TGWLDYLIAEKVQAEKPDIMLGV | (SEQ ID NO:6) |
| | ***** ** ** * * * | |

As an additional example, the design of constructs for expression of other ACC BC domains can be based on homology to the U. maydis BC domain of pCS8 as shown in FIG. 10 of PCT Application WO 2004/013159 and in Table 1B below.

TABLE 1B
Alignment of fungal ACCase BC Domains

| | |
|---|---|
| ustilago | ------------------------PPPD--------HKAVSQ-----------FIG-GNP |
| phytophthora | -VAEEAP-----------------PAAD--------VAAYAE-----------TRSDSNP |
| yeast | SEESLFESS---------------PQKM--------EYEITNYSERHTELPGHFIG-LNT |
| magnaporthe | TETNGTAAAANSSRQRNGANGVTVPVANGKATYAQRHKIADH-----------FIG-GNR |
| | * * |
| | y |
| ustilago | LETAPASPVADFIRKQGGHSVITKVLICNNGIAAVKEIRSIRKWAYETFGDERAIEFTVM |
| phytophthora | LNYA---SMEEYVRLQKGTRPITSVLIANNGISAVKAIRSIRSWSYEMFADEHVVTFVVM |
| yeast | VDKLEESPLRDFVKSHGGHTVISKILIANNGIAAVKEIRSVRKWAYETFGDDRTVQFVAM |
| magnaporthe | LENAPPSKVKEWVAAHDGHTVITNVLIANNGIAAVKEIRSVRKWAYETFGDERAIQFTVM |
| | * * ** **** *** *** * * ** * * * * |
| ustilago | ATPEDLKVNADYIRMADQYVEVPGGSNNNNYANVDLIVDVAERAGVHAVWAGWGHASENP |
| phytophthora | ATPEDLKANAEYIRMAEHVVEVPGGSNNHNYANVSLIIEIAERFNVDAVWAGWGHASENP |
| yeast | ATPEDLEANAEYIRMADQYIEVPGGTNNNNYANVDLIVDIAERADVDAVWAGWGHASENP |
| magnaporthe | ATPEDLQANADYIRMADHYVEVPGGTNNNNYANVELIVDVAERMNVHAVWAGWGHASENP |
| | ****** ** ***** ***** ** ***** ** *** * ************* |
| ustilago | RLPESLAASKHKIIFIGPPGSAMRSLGDKISSTIVAQHADVPCMPWSGTGIKETMMSDQ- |
| phytophthora | LLPDTLAQTERKIVFIGPPGKPMRALGDKIGSTIIAQSAKVPTIAWNGDGMEVDYKEHD- |
| yeast | LLPEKLSQSKRKVIFIGPPGNAMRSLGDKISSTIVAQSAKVPCIPWSGTGV-DTVHVDEK |
| magnaporthe | KLPESLAASPKKIIFIGPPGSAMRSLGDKISSTIVAQHAQVPCIPWSGTGVDAVQIDKK- |
| | ** * * ****** ** ***** *** ** * ** * * * |
| ustilago | -GFLTVSDDVYQQACIHTAEEGLEKAEKIGYPVMIKASEGGGGKGIRKCTNGEEFKQLYN |
| phytophthora | -G---IPDEIYNAAMLRDGQHCLDECKRIGFPVMIKASEGGGGKGIRMVHEESQVLSAWE |
| yeast | TGLVSVDDDIYQKGCCTSPEDGLQKAKRIGFPVMIKASEGGGGKGIRQVEREEDFIALYH |
| magnaporthe | -GIVTVDDDTYAKGCVTSWQEGLEKARQIGFPVMIKASEGGGGKGIRKAVSEEGFEELYK |
| | * * * * ** **************** |
| ustilago | AVLGEVPGSPVFVMKLAGQARHLEVQLLADQYGNAISIFGRDCSVQRRHQKIIEEAPVTI |
| phytophthora | AVRGEIPGSPIFVMKLAPKSRHLEVQLLADTYGNAIALSGRDCSVQRRHQKIVEEGPVLA |
| yeast | QAANEIPGSPTEIMKLAGRARHLEVQLLADQYGTNISLFGRDCSVQRRHQKIIEEAPVTI |
| magnaporthe | AAASEIPGSPIFIMKLAGNARHLEVQLLADQYGNNISLFGRDCSVQRRHQKIIEEAPVTI |
| | * **** * **** ********** ** * ************* ** ** |
| ustilago | APEDARESMEKAAVRLAKLVGYVSAGTVEWLYS--PESG--EFAFLELNPRLQVEHPTTE |
| phytophthora | PTQEVWEKMMPAATRLAQEVEYVNAGTVEYLFSELPEDNGNSFFFLELNPRLQVEHPVTE |
| yeast | AKAETFHEMEKAAVRLGKLVGYVSAGTVEYLYS--HDDG--KFYFLELNPRLQVEHPTTE |
| magnaporthe | AKPDTFKAMEEAAVRLGRLVGYVSAGTVEYLYS--HADD--KFYFLELNPRLQVEHPTTE |
| | * ** ** * ** ***** * * * ************* ** |
| ustilago | MVSGVNIPAAQLQVAMGIPLYSIRDIRTLYGMDPRGNEVIDFDFSSPESFKTQRKPQPQG |
| phytophthora | MITHVNLPAAQLQVAMGIPLHCIPDVRRLYNKDAFETTVIDFD--------AEKQKPPHG |
| yeast | MVSGVNLPAAQLQIAMGIPMHRISDIRTLYGMNPHSASEIDFEFKTQDATKKQRRPIPKG |
| magnaporthe | GVSGVNLPASQLQIAMGIPLHRISDIRLLYGVDPKLSTEIDFDFKNPDSEKTQRRPSPKG |
| | ** ** *** ***** * * * ** *** * * |
| ustilago | HVVACRITAENPDTGFKPGMGALTELNFRSSTSTWGYFSVGTSGALHEYADSQFGHIFAY |
| phytophthora | HVIAARITAEDPNAGFQPTSGAIQELNFRSTPDVWGYFSVDSSGQVHEFADSQTGHLFSW |
| yeast | HCTACRITSEDPNDGFKPSGGTLHELNFRSSSNVWGYFSVGNNGNIHSFSDSQFGIUFAF |
| magnaporthe | HLTACRITSEDPGEGRKPSNGVMHELNFRSSSNVWGYFSVGTQGGIHSFSDSQFGHIFAY |
| | * * *** * * ** * * ****** ****** * * *** ** * |
| ustilago | GADRSEARKQMVISLKELSIRGDFRTTVEYLIKLLETDAFESNKITTGWLDGLIQDRLTA |
| phytophthora | SPTREKARKNMVLALKELSIRGDIHTTVEYIVNNMESDDFKYNRISTSWLDERTSHHNEV |
| yeast | GENRQASRKHMVVALKELSIRGDFRTTVEYLIKLLETEDFEDNTITTGWLDDLITHKMTA |
| magnaporthe | GENRSASRKHMVIALKELSIRGDFRTTVEYLIKLLETEAFEENTITTGWLDELISKKLTA |
| | * ** ** ********* ***** * * * * * *** * |

| | | |
|---|---|---|
| ustilago | E--RPPADLAV | (SEQ ID NO:4) |
| phytophthora | RLQGRPD----- | (SEQ ID NO:7) |
| yeast | E---KPDPTLAV | (SEQ ID NO:8) |
| magnaporthe | E---RPDKMLAV | (SEQ ID NO:9) |
| | * | |

The methods, storage media, data structures, and the like, along with compounds identified by such methods and methods of use thereof, may be implemented in like manner as described in L. Tong et al., PCT Application WO 2004/063715, titled Methods of Using Crystal Structure of Carboxyltransferase Domain of Acetyl-CoA Carboxylase, Modulators Thereof, and Computer Methods.

The present invention provides for methods of using a computer to identify modulators of a target BC domain of ACC comprising using a computer-readable three-dimensional structure of the BC domain of an ACC enzyme, a substrate or modulator binding site of the BC domain of ACC, and/or an active site of the BC domain of ACC to design and/or select for a potential modulator of the BC domain of ACC based on the predicted ability of the modulator to bind to a binding site, for example, of the BC domain of ACC. The invention fluther provides for synthesizing and testing the designed or selected modulator for its ability to modulate the activity of the target BC domain of ACC. For example, a potential modulator may be contacted with the target enzyme in the presence of one or more substrates, and the ability of the target enzyme to act on its substrate in the presence or absence of potential modulator may be measured and compared. As another specific, non-limiting example, the designed or selected potential modulator may be synthesized and introduced into an in vivo or in vitro model system and then the production of malonyl-CoA may be monitored. A modulator that decreases the relative amount of malonyl-CoA may be useful in the treatment of obesity, metabolic syndrome, diabetes, cardiovascular disease, atherosclerosis and infections, whereas a modulator that increases malonyl-CoA may be useful to promote endurance or survival in stressful conditions. In one embodiment, the modulator decreases the activity of ACC2 but not ACC1. In another embodiment, the modulator decreases the activity of both ACC1 and ACC2 resulting in increased fatty acid oxidation in oxidative tissue and reduced fatty acid synthesis in lipogenic tissue thus preventing any compensatory effects (Harwood, H. J. et al. (2003). J Biol Chem 278, 37099-37111). A modulator can be essentially any compound, including, a small-molecule, a peptide, a protein, a nucleic acid (including siRNA, anti-sense RNA, catalytic DNA or RNA, DNAzymes, Ribozymes) and antibodies and antibody fragments.

Modulators identified according to the instant invention also may be used as fungicides, insecticides or herbicides. In a further specific, non-limiting example, a designed or selected potential modulator may be contacted with the target enzyme in the presence of a known inhibitor that binds to the BC domain of ACC (i.e., soraphen) to determine whether the potential modulator competes for binding of the inhibitor. The potential modulator also may be tested for its ability to inhibit the growth of certain organisms (i.e., fungi, insects, plants), and the potential modulator may selectively inhibit the growth of undesirable organisms such as pathogenic fungi, insect pests or weeds. Because the acetyl-CoA carboxylase molecule is large, it is very difficult to crystallize, and has not yet been crystallized. This invention, therefore, provides a solution to a long-felt need, for providing a method to rationally design or modify compounds known to bind to ACC. The provided structure of the BC domain of ACC only now enables one to define, and therefore adjust, the binding mode of any given compound. The virtual models, atomic structure, methods and compositions provided by this invention are useful in the drug discovery of further, as yet unindentified inhibitors or modulators of ACC, and in the design or redesign of modulators of ACC activity.

The present invention also provides for molecules which comprise binding site(s) and/or active sites of the BC domain of ACC, as defined by the atomic coordinates provided by the present invention, in an otherwise synthetic molecule. Such a molecule may be used to screen test compounds, for example compounds in a combinatorial library, for binding to the active site and/or binding sites and/or for suitability as ligands. Within the present invention, a binding site of the BC domain can also be referred to as a binding cavity or a binding pocket. Further, in the present invention, a ligand of a BC domain encompasses essentially any molecule that can bind to the BC domain, including a substrate or a modulator.

The present invention further provides for a method of designing or selecting an inhibitor or agonist of ACC comprising creating a computer model of the negative space present in an unoccupied binding site and/or active site of the BC domain of ACC, which can take into account the electron densities at the boundaries of this space, and using such a model to design or select molecules that modulate the activity of ACC. Such a negative space, particularly a space presented in the context of electrophilic and electrophobic boundaries, in computer readable, electronic form, stored or storable on a floppy disc or computer hard drive, may provide a simple template for the design and/or selection of modulator compounds.

In addition, the present invention provides for a method of evaluating the binding properties of a potential modulator comprising co-crystallizing the modulator with the BC domain of ACC, determining the three-dimensional structure of the modulator bound to the BC domain of ACC and analyzing the three-dimensional structure of the BC domain of ACC bound to the modulator to evaluate the structural aspects of binding. Such a structure may further be used to design and/or select improved potential modulator compounds.

In another embodiment, the present invention provides for polynucleotides encoding an ACC polypeptide having a mutation in one or more residues of the soraphen binding site. Further, BC domain polynucleotides are useful, inter alia, for producing herbicide resistant plants. Accordingly, the present invention also relates to genetically modified herbicide resistant plants.

The present invention further provides for an isolated and purified peptide fragment comprising the BC domain of ACC. In one embodiment, a BC domain of ACC is that provided by the ACC yeast (yACC) construct, pCS16. The isolated and purified peptide fragment comprising the BC domain of ACC is useful, inter alia, for the screening and assay of compounds which modulate the activity of the BC domain of ACC. As noted supra, modulators of the BC domain of ACC may be used in the treatment of various diseases and disorders, including but not limited to, obesity, metabolic syndrome, diabetes, cardiovascular disease, atherosclerosis and infections. The isolated and purified peptide fragment comprising the BC domain of ACC also may be used to design and/or screen metabolic enhancers that may be used to promote endurance or survival under stressful conditions.

The modulators of the activity of the BC domain of ACC to be screened or assayed using the isolated and purified BC domain of ACC of the instant invention may be those designed or identified using the crystal structures concerning the BC domain of ACC provided herein, or they may be existing compounds not previously known to be modulators of the BC domain of ACC.

In one embodiment, the present invention encompasses allelic variants and mutations of the BC domain sequences disclosed herein that are at least 85 percent, at least 90 percent, or at least 95 percent homologous to the naturally occurring BC domain, with homology being determined by standard computer software, such as BLASTP, or ClustalW used with a scoring matrix such as BLOSUM or PAM.

A modulator of ACC enzyme activity refers to a compound which can alter the amount of product generated by a reaction catalyzed by the enzyme. The alteration may be an increase or a decrease. A compound that increases the amount of product is considered an agonist and a compound that decreases the amount of product is considered an inhibitor. Where the biological function of an enzyme encompasses both directions of a reaction (for example ACC catalyzes the carboxylation of acetyl-CoA to produce malonyl-CoA and the decarboxylation of malonyl-CoA to produce acetyl-CoA), whether a modulator is acting as an agonist or an inhibitor depends upon the amount of malonyl-CoA produced. A modulator which decreases the production of malonyl-CoA is an inhibitor. A decrease in malonyl-CoA results in an increase in fatty acid oxidation and a decrease in fatty acid synthesis. Such a decrease may be useful for the treatment of obesity, metabolic syndrome, diabetes, cardiovascular disease, atherosclerosis and infections.

A substrate binding site refers to a region of the BC domain of ACC that retains substrate (for example, biotin) in a position suitable for carboxylation to occur. The configuration of the substrate binding site is likely to be different in the presence and absence of bound substrate, and both configurations are optimally considered in the design and/or selection of enzyme modulators.

Determination of Crystal Structure

The three-dimensional structure of a BC domain of ACC may be determined by obtaining its crystal structure directly and/or by comparing the primary and/or secondary structure of the BC domain of ACC, and/or an incomplete set of components of its three-dimensional structure, with a crystal structure that has already been solved.

The three-dimensional structures obtained from crystals of the BC domain of yeast ACC (“yACC”) and the BC domain in complex with the modulator soraphen, may be employed to solve the structures of the BC domains of other ACC species, including but not limited to the BC domains of Magnaporthe (mgACC), Ustilago maydis (uACC), Phytophorthora infestans (pACC), human ACC (hACC: ACC1 and ACC2) and mouse ACC (mACC), as well as the structures of the BC domains of other acetyl-CoA carboxylases.

The BC domain of ACC may be prepared from a natural source, may be produced by recombinant DNA technology, or may be chemically synthesized (although this last possibility would be extremely cumbersome). For example, a full-length cDNA encoding an acetyl-CoA carboxylase such as ACC may be subcloned from a cDNA preparation by the polymerase chain reaction (PCR), using at least one primer design based on known, homologous, or obtained protein sequence, and inserted into an expression vector. Standard deletion mutagenesis techniques then may be used to remove those regions of the ACC cDNA not encoding the BC domain.

A nucleic acid encoding a BC domain of ACC, or a fusion protein comprising said BC domain of ACC, may be operably linked to other elements which aid in its expression, such as a promoter element. One of skill in the art would know, based on the degeneracy of the genetic code, how to set out the many possible nucleotide sequences that would code for the amino acids of BC domains. A large number of suitable vector-host systems are known in the art. Possible vectors include, but are not limited to, plasmids or modified viruses, but the vector system must be compatible with the host cell used. Examples of vectors include E. coli bacteriophages such as lambda derivatives, or plasmids such as pBR322 derivatives or pUC plasmid derivatives, e.g., pGEX vectors (Amersham-Pharmacia, Piscataway, N.J.), pET vectors (Novagen, Madison, Wis.), pmal-c vectors (Amersham-Pharmacia, Piscataway, N.J.), pFLAG vectors (Chiang and Roeder, 1993, Pept. Res. 6:62-64), baculovirus vectors (Invitrogen, Carlsbad, Calif.; Pharmingen, San Diego, Calif.), etc. The insertion into a cloning vector can, for example, be accomplished by ligating the DNA fragment into a cloning vector which has complementary cohesive termini, by blunt end ligation if no complementary cohesive termini are available or through nucleotide linkers using techniques standard in the art. E.g., Ausubel et al. (eds.), Current Protocols in Molecular Biology, (1992). Recombinant vectors comprising the nucleic acid of interest may then be introduced into a host cell compatible with the vector (e.g. E. coli, insect cells, mammalian cells, etc.) via transformation, transfection, infection, electroporation, etc. The nucleic acid may also be placed in a shuttle vector which may be cloned and propagated to large quantities in bacteria and then introduced into a eukaryotic host cell for expression. The vector systems of the present invention may provide expression control sequences and may allow for the expression of proteins in vitro.

The BC domains of any of the afore-mentioned ACCs, produced either naturally, synthetically or by recombinant means, may be purified by methods known in the art, including, but not limited to, selective precipitation, dialysis, chromatography, and/or electrophoresis. Purification may be monitored by measuring the ability of a fraction to perform the catalytic activity. Any standard method of measuring acetyl-CoA carboxylase activity may be used.

For certain embodiments, it may be desirable to express the BC domain of ACC as a fusion protein. In specific non-limiting embodiments, the fusion protein comprises a tag which facilitates purification. As referred to herein, a “tag” is any added series of amino acids which are provided in a protein at either the C-terminus, the N-terminus, or internally. Suitable tags include but are not limited to tags known to those skilled in the art to be useful in purification such as, but not limited to, His tag, glutathione-s-transferase tag, flag tag, mbp (maltose binding protein) tag, etc. Such tagged proteins may also be engineered to comprise a cleavage site, such as a thrombin, enterokinase or factor X cleavage site, for ease of removal of the tag before, during or after purification. Vector systems which provide a tag and a cleavage site for removal of the tag are particularly useful to make the expression constructs of the present invention. A tagged ACC may be purified by immuno-affinity or conventional chromatography, including but not limited to, chromatography employing the following: glutathione-Sepharose™ (Amersham-Pharmacia, Piscataway, N.J.) or an equivalent resin, nickel or cobalt-purification resins, nickel-agarose resin, anion exchange chromatography, cation exchange chromatography, hydrophobic resins, gel filtration, antiflag epitope resin, reverse phase chromatography, etc.

Any crystallization technique known to those skilled in the art may be employed to obtain the crystals of the present invention, including, but not limited to, batch crystallization, vapor diffusion (either by sitting drop or hanging drop) and micro dialysis. Seeding of the crystals in some instances may be required to obtain X-ray quality crystals. Standard micro and/or macro seeding of crystals may therefore be used. In one embodiment, the crystals are obtained using the sitting-drop vapor diffusion method. Different crystallization methods can result in the formation of different crystal forms (i.e., polymorphs or solvates), and thus, the present invention encompasses the different crystal forms for the BC domain of ACC.

To collect diffraction data from the crystals of the present invention, the crystals may be flash-frozen in the crystallization buffer employed for the growth of said crystals, however with preferably higher precipitant concentration (see, Examples below). For example, but not by way of limitation, if the precipitant used was 20% PEG 3350, the crystals may be flash frozen in the same crystallization solution employed for the crystal growth wherein the concentration of the precipitant is increased to 25% (see Examples below). If the precipitant is not a sufficient cryoprotectant (i.e. a glass is not formed upon flash-freezing), cryoprotectants (e.g. glycerol, ethylene glycol, low molecular weight PEGs, alcohols, etc.) may be added to the solution in order to achieve glass formation—upon flash-freezing, providing the cryoprotectant is compatible with preserving the integrity of the crystals. The flash-frozen crystals are maintained at a temperature of less than −110° C. or less than −150° C. during the collection of the crystallographic data by X-ray diffraction.

In certain embodiments, the protein crystals and protein-substrate complex co-crystals of the present invention diffract to a high resolution limit of at least greater than or equal to 3.5 angstrom (Å) or greater than or equal to 3 Å; it should be noted that a greater resolution is associated with the ability to distinguish atoms placed closer together. In one embodiment, the protein crystals and protein-substrate complex co-crystals of the present invention diffract to a high resolution limit of greater than 2.5 Å or 1.5 Å.

Data obtained from the diffraction pattern may be solved directly or may be solved by comparing it to a known structure, for example, the three-dimensional structure of the BC domain of yACC (with or without substrates or modulators). If the crystals are in a different space group than the known structure, molecular replacement may be employed to solve the structure, or if the crystals are in the same space group, refinement and difference Fourier methods may be employed. The structure of the BC domain of ACC, as defined herein, exhibits no greater than about 4.0 Å, 1.5 Å or 0.5Å root mean square deviation (RMSD) in the positions of the Cα atoms for at least 50% or more of the amino acids.

Any method known to those skilled in the art may be used to process the X-ray diffraction data. In addition, in order to determine the atomic structure of an ACC according to the present invention, multiple isomorphous replacement (MIR) analysis, model building and refinement may be performed. For MIR analysis, the crystals may be soaked in heavy-atoms to produce heavy atom derivatives necessary for MIR analysis. As used herein, heavy atom derivative or derivatization refers to the method of producing a chemically modified form of a protein or protein complex crystal wherein said protein is specifically bound to a heavy atom within the crystal. In practice a crystal is soaked in a solution containing heavy metal atoms or salts, or organometallic compounds, e.g., lead chloride, gold cyanide, thimerosal, lead acetate, uranyl acetate, mercury chloride, gold chloride, etc., which can diffuse through the crystal and bind specifically to the protein. The location(s) of the bound heavy metal atom(s) or salts can be determined by X-ray diffraction analysis of the soaked crystal. This information is used to generate MIR phase information which is used to construct the three-dimensional structure of the crystallized BC domain of ACC of the present invention. Thereafter, an initial model of the three-dimensional structure may be built using the program O (Jones et al., 1991, Acta Crystallogr. A47:110-119). The interpretation and building of the structure may be further facilitated by use of the program CNS (Brunger et al., 1998, Acta Crystallogr. D54:905-921).

The method of molecular replacement broadly refers to a method that involves generating a preliminary model of the three-dimensional structure of crystal of a BC domain of an ACC of the present invention whose structural coordinates were previously unknown. Molecular replacement is achieved by orienting and positioning a molecule whose structural coordinates are known (e.g. BC domain of yeast ACC, yACC, as described herein) within the unit cell as defined by the X-ray diffraction pattern obtained from the BC domain of an ACC under study (or the corresponding enzyme/substrate complex or enzyme/inhibitor complex) so as to best account for the observed diffraction pattern of the unknown crystal. Phases can then be calculated from this model and combined with the observed amplitudes to give an approximate Fourier synthesis of the structure whose coordinates are unknown. This in turn can be subject to any of several forms of refinement to provide a final, accurate structure.

The molecular replacement method may be applied using techniques known to the skilled artisan.

The three-dimensional structures and the specific atomic coordinates associated with said structures of the BC domain of yeast ACC, alone or in complex with a substrate such as acetyl-CoA or a modulator, are useful for solving the structure of crystallized forms of BC domains of other ACCs. This technique may could also be applied to solve the structures of ACC-related proteins, where there is sufficient sequence identity. Such ACC-related proteins comprise a root mean square deviation (RMSD) of no greater than 2.0 Å, 1.5 Å, 1.0 Å or 0.5 Å in the positions of Cα atoms for at least 50 percent or more of the amino acids of the structure of the BC domain of ACC of the present invention. Such an RMSD may be expected based on the amino acid sequence identity. Chothia and Lesk, 1986, EMBO J 5:823-826.

Design of Modulators

Modulators of ACC may be designed, according to the invention, using three-dimensional structures obtained as set forth in the preceding section and the Examples section below. These structures may be used to design or screen for molecules that are able to form the desired interactions with one or more binding sites of the BC domain of ACC.

The models of the BC domain (and sub-regions, including active sites, binding sites or cavities thereof) of ACC described herein may be used to either directly develop a modulator for ACC or indirectly develop a modulator of an ACC-related enzyme for which the structure has not yet been solved. A modulator designed to interact with a BC domain may be reasonably expected to interact not only with the BC domain but may also interact with BC domains isolated from other organisms. The ability for such a modulator to modulate the activity of a BC domain of ACC can be confirmed by further computer analysis, and/or by in vitro and/or in vivo testing.

In non-limiting embodiments, the present invention provides for a model, actual or virtual, of the BC domain (the whole domain, or parts, such as a particular substrate or modulator binding site) of ACC.

A model of an active site may be comprised in a virtual or actual protein structure that is smaller than, larger than, or the same size as a native BC domain of an ACC protein. The protein environment surrounding the active site model may be homologous or identical to native BC domain of an ACC, or it may be partially or completely non-homologous.

Thus, the present invention provides for a method for rationally designing a modulator of an ACC, comprising the steps of (i) producing a computer readable model of a molecule comprising a region (i.e., an active site, reactive site, or a binding site) of a BC domain of ACC (e.g. yACC); and (ii) using the model to design a test compound having a structure and a charge distribution compatible with (i.e. able to be accommodated within) the region of the BC domain, wherein the test compound can comprise a functional group that may interact with the active site to modulate acetyl-CoA carboxylase activity. If the crystal structure is not available for the BC domain to be examined, homology modeling methods known to those of ordinary skill in the art may be used to produce a model, which then may be used to design test compounds as described above.

The atomic coordinates of atoms of the BC domain (or a region/portion thereof) of an ACC or an ACC-related enzyme may be used in conjunction with computer modeling using a docking program such as GRAM, DOCK, HOOK or AUTODOCK (Dunbrack et al., 1997, Folding & Design 2:27-42) to identify potential modulators. This procedure can include computer fitting of potential modulators to a model of a BC domain (including models of regions of a BC domain, for example, an active site, or a binding site) to ascertain how well the shape and the chemical structure of the potential modulator will complement the active site or to compare the potential modulators with the binding of substrate or known inhibitor molecules in the active site.

Computer programs may be employed to estimate the attraction, repulsion and/or steric hindrance associated with a postulated interaction between the reactive site model and the potential modulator compound. Generally, characteristics of an interaction that are associated with modulator activity include, but are not limited to, tight fit, low steric hindrance, positive attractive forces, and specificity.

Modulator compounds of the present invention may also be designed by visually inspecting the three-dimensional structure of a reactive site of the BC domain of an ACC or ACC-related enzymes, a technique known in the art as “manual” drug design. Manual drug design may employ visual inspection and analysis using a graphics visualization program known in the art.

In designing potential modulator compounds according to the invention, the functional aspect of a modulator may be directed at a particular step of the ACC catalytic mechanism, as illustrated by the following non-limiting example.

Screening for Modulator Compounds

As an alternative or an adjunct to rationally designing modulators, random screening of a small molecule library, a peptide library or a phage library for compounds that interact with and/or bind to a site/region of interest (i.e., a binding site, active site or a reactive site, for example) of the BC domain of ACC or ACC-related enzymes may be used to identify useful compounds. Such screening may be virtual; small molecule databases can be computationally screened for chemical entities or compounds that can bind to or otherwise interact with a virtual model of an active site, binding site or reactive site of a BC domain of an ACC. Alternatively, screening can be against actual molecular models of the BC domain or portions thereof. In one embodiment, modulators which selectively bind ACC2 and not ACC1, or vice versa, are screened. In another embodiment, modulators which selectively bind to yeast ACC and not human ACC1 or ACC2 are screened. Further, antibodies can be generated that bind to a site of interest of the BC domain. After candidate (or “test”) compounds that can bind to the BC domain are identified, the compounds can then be tested to determine whether they can modulate BC domain enzymatic activity (see Assay Systems section below).

In one embodiment, BC domain proteins, nucleic acids, and cells containing the BC domains are used in screening assays. Screens may be designed to first find candidate compounds that can bind to a BC domain or portion thereof, and then these compounds may be used in assays that evaluate the ability of the candidate compound to modulate BC domain or ACC enzymatic activity. Thus, as will be appreciated by those in the art, there are a number of different assays which may be run, including binding assays and activity assays. In one aspect, candidate compounds are first tested to determine whether they can bind to a particular binding site of the BC domain.

Thus, in one embodiment, the methods comprise combining a BC domain or portion thereof and a candidate compound, and determining the binding of the candidate compound to the BC domain or portion thereof. In some embodiments of the methods herein, the BC domain (or portion thereof), or possibly the candidate agent, is non-diffusably bound to an insoluble support having isolated sample receiving areas (e.g., a microtiter plate, an array, etc.). The insoluble supports may be made of any composition to which the compositions can be bound, is readily separated from soluble material, and is otherwise compatible with the overall method of screening. The surface of such supports may be solid or porous and of any convenient shape. Examples of suitable insoluble supports include microtiter plates, arrays, membranes and beads. These are typically made of glass, plastic (e.g., polystyrene), polysaccharides, nylon or nitrocellulose, Teflon™, etc. Microtiter plates and arrays are especially convenient because a large number of assays can be carried out simultaneously, using small amounts of reagents and samples—i.e., they enable high-throuput screening. Following binding of the BC domain, excess unbound material is removed by washing. The sample receiving areas may then be blocked through incubation with bovine serum albumin (BSA), casein or other innocuous protein or other moiety.

A candidate compound is added to the assay. Candidate compounds include, but are not limited to, specific antibodies, compounds from chemical libraries, peptide analogs, etc. Of particular interest are screening assays for compounds that have a low toxicity for human cells. A wide variety of assays may be used for this purpose, including labeled in vitro protein-protein binding assays, immunoassays for protein binding, NMR assays to determine protein-protein or protein-chemical compound binding, and the like. Candidate compounds can also include insecticides, herbicides or fungicides.

The term “candidate compound” as used herein describes any molecule, e.g., protein, oligopeptide, small organic molecule, polysaccharide, polynucleotide, etc., with the capability of directly or indirectly modulating BC domain or ACC enzymatic activity. Generally a plurality of assay mixtures are run in parallel with different compound concentrations to obtain a differential response to the various concentrations. Typically, one of these concentrations serves as a negative control, i.e., at zero concentration or below the level of detection.

Candidate compounds can encompass numerous chemical classes, though typically they are organic molecules, and in one embodiment they are small organic compounds having a molecular weight of more than 100 and less than about 2,500 daltons. Candidate compounds can comprise functional groups necessary for structural interaction with proteins, for example hydrogen bonding, and typically include at least an amine, carbonyl, hydroxyl or carboxyl group, preferably at least two of the functional chemical groups. The candidate compounds can comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Particularly preferred candidate compounds are those having the characteristics of “example modulators” as described below.

Candidate compounds can be obtained from a wide variety of sources including libraries of synthetic or natural compounds. For example, numerous means are available for random and directed synthesis of a wide variety of organic compounds and biomolecules, including combinatorial chemical synthesis and the expression of randomized peptides or oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs. In one embodiment, the library is fully randomized, with no sequence preferences or constants at any position. In another, the library is biased. That is, some positions within the sequence are either held constant, or are selected from a limited number of possibilities.

The determination of the binding of the candidate compound to the BC domain may be done in a number of ways. In one embodiment, the candidate compound is labelled, and binding determined directly. For example, this may be done by attaching all or a portion of the BC domain to a solid support, adding a labelled candidate compound (for example a fluorescent label or radioactive label), washing off excess reagent, and determining whether the label is present on the solid support. Various blocking and washing steps may be utilized as is known in the art.

By “labeled” herein is meant that the compound is either directly or indirectly labelled with a label which provides a detectable signal, e.g., radioisotope, fluorescers, enzyme, antibodies, particles such as magnetic particles, chemiluminescers, or specific binding molecules, etc. Specific binding molecules include pairs, such as biotin and streptavidin, digoxin and antidigoxin, etc. For the specific binding members, the complementary member would normally be labeled with a molecule which provides for detection, in accordance with known procedures, as outlined above. The label can directly or indirectly provide a detectable signal.

In one embodiment, the binding of the candidate compound is determined through the use of competitive binding assays. In this embodiment, the competitor is a binding moiety known to bind to the BC domain, such as an antibody, peptide, ligand (i.e., soraphen), etc. Under certain circumstances, there may be competitive binding as between the candidate compound and the known binding moiety, with the binding moiety displacing the bioactive agent.

In one embodiment, the candidate compound is labeled. Either the candidate compound, or the competitor, or both, is added first to the BC domain for a time sufficient to allow binding, if present. Incubations may be performed at any temperature which facilitates optimal binding, typically between 4 and 40° C. Incubation periods are selected for optimum binding but may also be optimized to facilitate rapid high-throughput screening. Typically between 0.1 and 1 hour will be sufficient. Excess reagent is generally removed or washed away. The second component is then added, and the presence or absence of the labeled component is followed, to indicate binding.

In one embodiment, the competitor is added first, followed by the candidate compound. Displacement of the competitor is an indication that the candidate compound is binding to the BC domain and thus is capable of binding to, and potentially modulating, the activity of the BC domain or ACC enzyme. In this embodiment, either component can be labeled. Thus, for example, if the competitor is labeled, the presence of label in the wash solution indicates displacement of the competitor by the candidate compound. Alternatively, if the candidate compound is labeled, the presence of the label on the support indicates displacement of the candidate compound.

In one embodiment, a potential ligand for a BC domain can be obtained by screening a recombinant bacteriophage library (Scott and Smith, Science, 249:386-390 (1990); Cwirla et al., Proc. Natl. Acad. Sci., 87:6378-6382 (1990); Devlin et al., Science, 249:404-406 (1990). Specifically, the phage library can be mixed in low dilutions with permissive E. coli in low melting point LB agar which is then poured on top of LB agar plates. After incubating the plates at 37° C. for a period of time, small clear plaques in a lawn of E. coli will form which represents active phage growth and lysis of the E. coli. A representative of these phages can be absorbed to nylon filters by placing dry filters onto the agar plates. The filters can be marked for orientation, removed, and placed in washing solutions to block any remaining absorbent sites. The filters can then be placed in a solution containing, for example, a radioactive BC domain (or portion thereof). After a specified incubation period, the filters can be thoroughly washed and developed for autoradiography. Plaques containing the phage that bind to the radioactive BC domain or portion thereof can then be identified. These phages can be further cloned and then retested for their ability to bind to the BC domain as before. Once the phages have been purified, the binding sequence contained within the phage can be determined by standard DNA sequencing techniques. Once the DNA sequence is known, synthetic peptides can be generated which represents these sequences, and firrther binding studies can be performed as discussed herein.

In another embodiment, a potential ligand for a BC domain can be obtained by screening candidate compounds by NMR (see for example, U.S. Patent Application Publication No. US2003/0148297A1 or Pellecchia et al., Nature Reviews Drug Discovery, 1:211-219 (2002)). As mentioned, a BC domain or portions thereof can be immobilized to all types of solid supports. It is not needed that the binding be a covalent binding. In the NMR measuring environment, the target may be in solution phase or may be immobilized. If immobilized, the target need not be directly immobilized to the solid support; it may also occur indirectly through suitable bridging moieties or molecules, or through spacers. Very suitable supports are solid polymers used in chromatography, such as polystyrene, sepharose and agarose resins and gels, e.g. in bead form or in a porous matrix form. Additionally, appropriately chemically modified silicon based materials are also very suitable supports.

Any soluble molecule can be used as a compound that is a candidate to binding to the BC domain. It is not necessary that the said soluble molecule is water-soluble. Any liquid medium that does not denature the said compound nor the BC domain molecule can be used in the NMR measurements. The BC domain target molecule is immobilized to a suitable support, such as a solid resin, and additionally placed in a suitable NMR probe, for example, a flow injection NMR probe, for the duration of the screening. Each sample of the compounds to be screened, e.g. the compounds from a library, is then applied to the immobilized target by pumping it through, along or via the solid support. The sample to be assayed may contain a single component suspected of binding to the BC domain target molecule, or may contain multiple components of a compound library or other type of collection or mixture. The flow may be stopped when a desired level of concentration of the compounds to be assayed is reached in the target containing probe or vessel.

For the acquisition of the NMR spectra, in principle any NMR pulse sequence capable of detecting resonances from dissolved molecule samples and, preferably suppressing residual solvent signals, such as by pulsed field gradients, may be used to detect binding. In practice, however, a one-dimensional 1H-NMR spectrum is acquired with sufficient resolution and sensitivity to detect and quantitate resonances derived from each compound being assayed in the presence of the control solid support. In addition, a second spectrum recorded using the same NMR protocol, is acquired for the same solution of screenable compounds in the presence of the solid support containing the immobilized BC domain target molecule. Optionally, a third spectrum may be acquired in the presence of the solid support containing the immobilized BC domain target molecule in order to detect extremely weak target binding. This spectrum can be recorded while using a diffusion or T2 filter.

After acquisition of the NMR spectrum, the sample of small compound or compounds is washed out of the NMR probe containing the target immobilized solid support. Subsequently, the next sample can be applied to the probe in a stopped-flow manner. Throughout the entire screening process a single sample of the target immobilized solid support remains in the NMR probe. The target immobilized solid support need only be changed should the target become denatured, chemically degraded or saturated by a tight-binding compound that cannot be washed away. In order to safeguard that certain compounds do not bind in such a way that the target molecule is blocked, at certain stages, a control is carried out to check the availability of binding opportunities to the target molecule.

The NMR spectra are preferably compared by subtracting one of the two NMR data sets from the other, thereby creating a difference spectrum. In general, since the target molecule is essentially in the solid phase, the resonances from compounds that bind to the target molecule are broadened beyond detection while in the bound state. Thus, binding is sensitively and reliably detectable by a decrease in height of peaks that derive exclusively from the solution form of compounds binding to the target molecule. This effect is most easily seen in the difference spectra. An alternative approach that can be used to quantitate the affinity of the target-ligand interaction is to determine peak areas (e.g. by integrating) in the control and experimental spectra and compare the values of these areas. Although it is possible to carry out the NMR screening method in batch mode, in the flow-injection set-up, one sample of target may be used to screen an entire library.

The present invention also encompasses antibodies that can specifically bind to the BC domain, including specific regions of the BC domain, such as binding sites. Antibodies include, for example, monoclonal antibodies and antibody fragments, such as Fab′, Fab, F(ab′)₂, single domain antibodies (DABs), Fv, and scFv (single chain Fv). The techniques for preparing and characterizing antibodies are well known in the art (see, for example, Antibodies: A Laboratory Manual, Cold Spring Harbor Laboratory, 1988). Monoclonal antibodies may be readily prepared through the use of well-known techniques, such as those exemplified in U.S. Pat. No. 4,196,265. Typically, this technique involves immunizing a suitable animal with a selected immunogen composition, e.g., a purified or partially purified ACC protein, ACC polypeptide, ACC peptide, BC domain or fragment thereof. The immunizing composition is administered in a manner effective to stimulate antibody-producing cells. These antibody-producing cells are then isolated and fused with tumor cells. The result of this cell fusion is a “hybridoma,” which will continually produce antibodies. These antibodies are called monoclonal because they come from only one type of cell, the hybridoma cell; polyclonal antibodies, on the other hand, are derived from preparations containing many kinds of cells.

Assay Systems

Potential modulators of acetyl-CoA carboxylase activity, produced, for example, by rational drug design or by screening of libraries as described above, may be subjected to one of the following assays to confirm their activity.

After identifying candidate compounds that can bind to the BC domain, these candidate compounds are then tested to determine whether they can modulate ACC enzymatic activity. For example, the candidate compounds can be tested by using enzyme kinetic assays to test the effects of a candidate compound upon BC domain catalytic activity.

A potential modulator may be subjected to virtual testing using a computer model of the BC domain of ACC or portions thereof, using the methods set forth for screening libraries of compounds. In other embodiments, a potential modulator may be evaluated for its ability to physically interact with the BC domain of an ACC or an ACC-related enzyme by co-crystallizing the potential modulator with the BC domain of the ACC or the ACC-related enzyme and then determining the structure of the resulting co-crystal. For example, the structure of the co-crystal may be determined by molecular replacement to assess the binding characteristics. The ability of the compound to modulate enzyme activity may be correlated with its ability to physically interact with the reactive site and/or to assume an orientation that would facilitate or inhibit carboxylation of malonyl.

The present invention further provides for assays comprising incubating the potential modulator with a purified BC domain of an ACC, such as yACC, MACC (ACC1 or ACC2) or hACC (ACC1 or ACC2) and determining the amount of acetyl carboxylation activity of the modulator-bound enzyme. To measure binding constants (e.g., K_{d}), methods known to those in the art may be employed such as Biacore™ analysis, isothermal titration calorimetry, fluorescence, ELISA with substrate on the plate to show competitive binding, or by a malonyl carboxylation activity assay. Similarly, the reaction rate may be measured by methods known in the art. In addition, relative binding affinities can be calculated, for example, to determine whether the modulator selectively binds ACC2 and not ACC 1.

The present invention further provides for methods that determine the effect of a potential modulator in vivo. Such methods may provide important information, including the effect of the modulator on molecules involved in interrelated pathways may be determined. For example, a potential modulator may be administered to a cell, such as a liver cell, a fat cell, a heart cell, or a skeletal cell, that is capable of regulating fatty acid oxidation, and/or the biosynthesis of long-chain fatty acids, and then the level of one or more molecules involved in fatty oxidation, the Embden-Meyerhoff pathway, the Krebs cycle, mitochondrial electron transport, fatty acid synthesis, and gluconeogenesis, including insulin, glycogen, cholesterol, and ketone bodies, may be measured, and the success or failure of the potential modulator to achieve the desired effect may be determined. For example, a modulator intended to effect preferential metabolism of fats (for example, in the treatment of obesity) may have one or more of the following effects: an increase in the acetyl-CoA/CoA ratio; increased intermediates or products of fatty acid oxidation; decreased intermediates or products of the Embden-Meyerhoff pathway, including lactic acid or lactate; decreased intermediates and products of fatty acid synthesis; decreased glycogen stores, increased ATP production, decreased ATP consumption, and decreased insulin sensitivity. The foregoing in vivo assays may be performed in a cell in the context of a cell culture, a tissue explant, and/or an organism. Equivalent in vitro systems that duplicate one or more of the recited pathways may also be used to assay the modulator for desired activity.

Further in vivo systems include plant in vivo systems in which the modulators of the present invention are administered to plants, and in particular crop plants, to determine whether the modulator is a potential fungicide. The ability to slow, cure or inhibit fungal growth indicates that the modulator is a candidate fungicide. Testing in a likewise manner as above for the ability of modulators to control insect pests or weedy pest would indicate that a modulator could be an insecticide or herbicide, respectively. Alternatively, a modulator may improve the growth of plants, in which case, the modulator may be useful as a growth enhancer. The modulators may also be tested for their ability to selectively slow or inhibit unwanted plant growth, while having a lesser effect on the herbicide resistant plants of the present invention.

Example Modulators

Modulators (also referred to as “active compounds” herein) that are identified by the methods described above are, in general, compounds: (i) having a molecular weight of from about 300 to 700, 800 or 1000 Kilodaltons, (ii) containing a ring system, optionally fused (e.g., two or three fused rings), of from 6 or 8 up to 20 atoms (which ring system may optionally contain 1, 2, 3, 4 or 5 or more hetero atoms selected from the group of N, O and S), (iii) optionally but preferably one, two or three additional cyclic groups (which may be cycloalkyl, heterocycloalkyl, aryl, or heteroaryl) linked to the ring system via a linking group; and (iv) optionally having one, two, three, or four or more additional substituents on the ring system and/or the additional cyclic group. Examples of such compounds include, but are not limited to:

(a) 1,4-Diazepine-2,5-diones, such as:

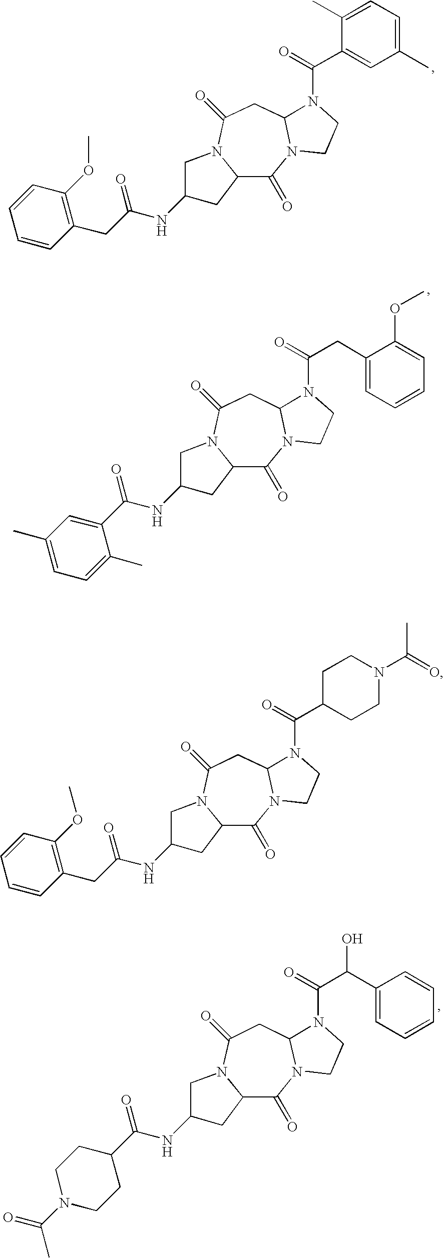

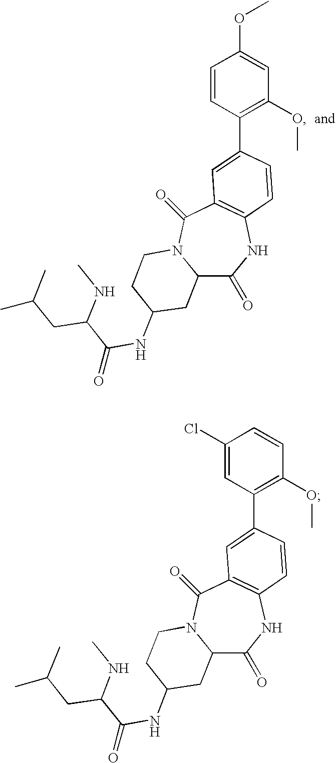

(b) Methyldecalins, such as:

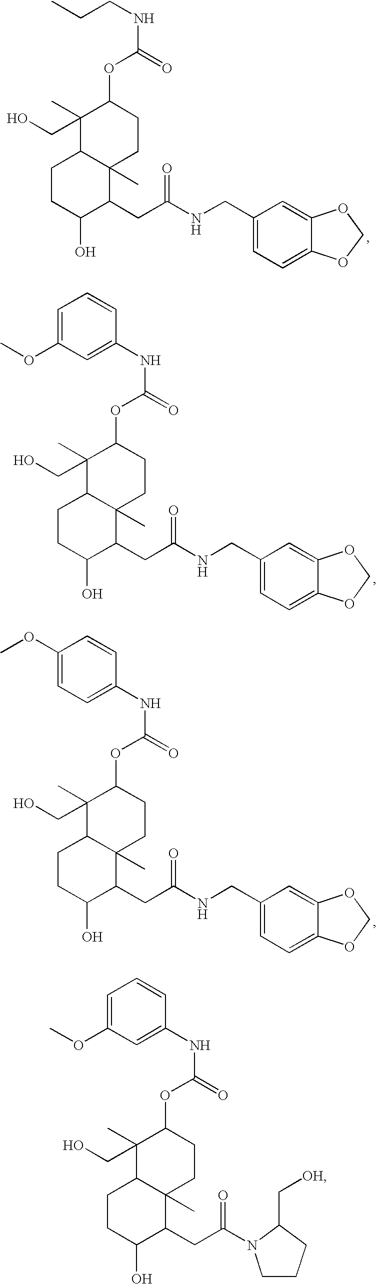

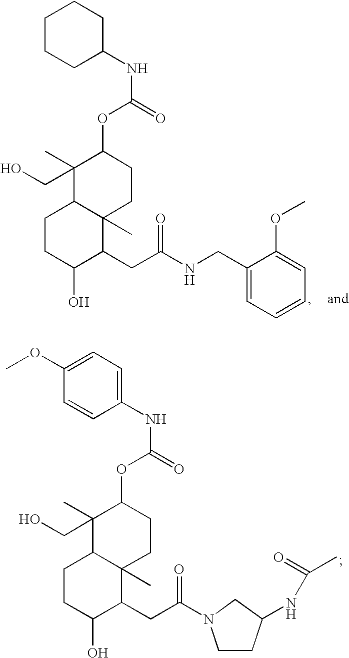

(c) Piperazine-2,5-diones, such as:

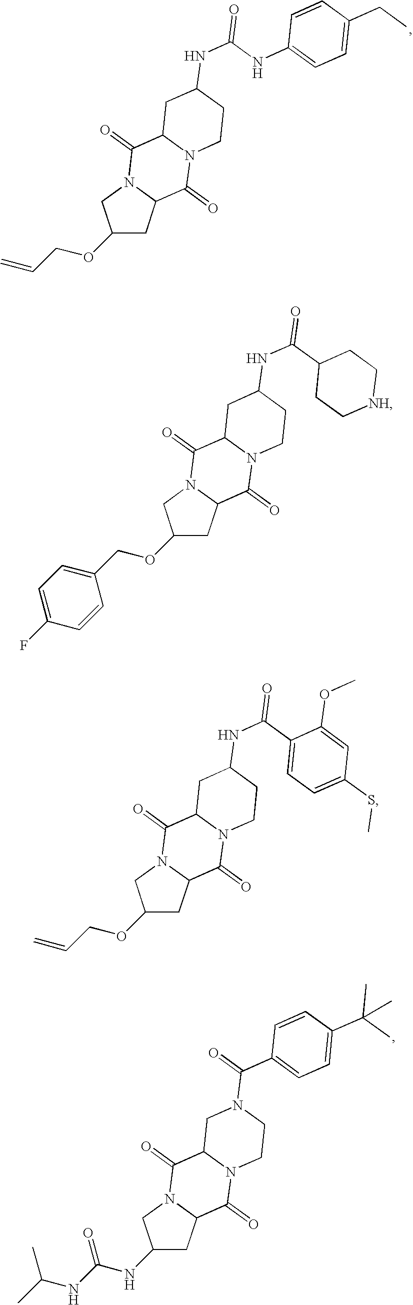

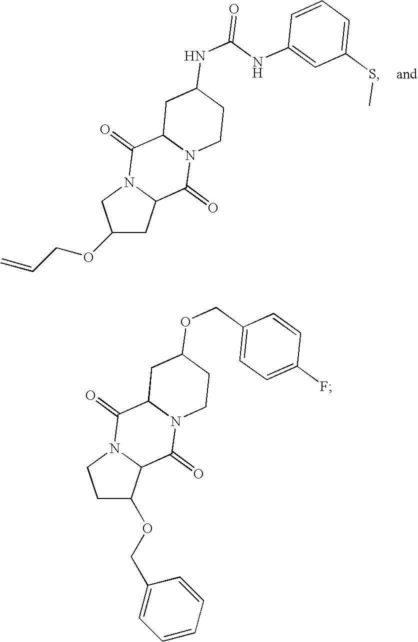

and (d) cytisines, such as:

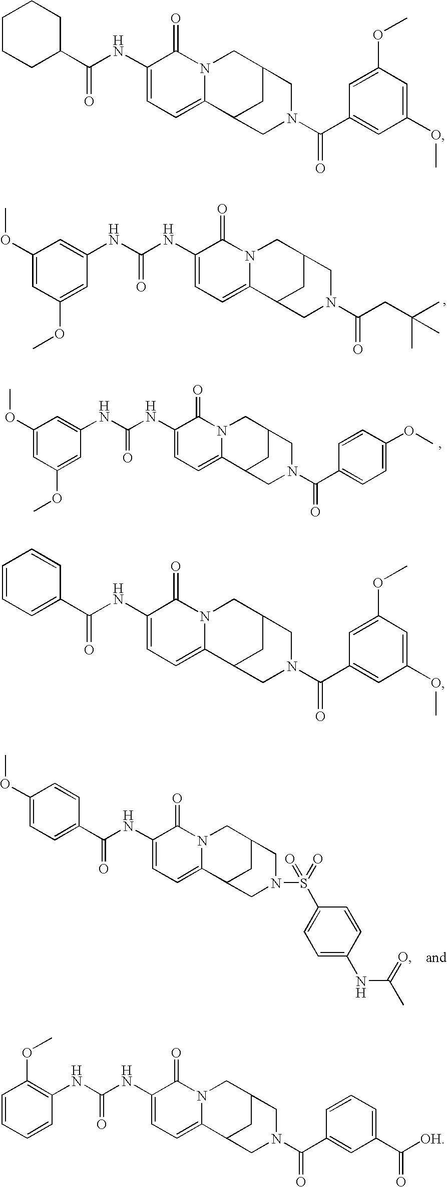

In some embodiments the compounds identified by the methods of the present invention are preferably not soraphen A or an analog thereof (e.g., preferably not macrocyclic polyketides), such as those compounds described in U.S. Pat. Nos. 5,026,878; 4,987,149; 4,954,517; and 4,940,804.

The compounds identified by the methods of the invention preferably competitively inhibits the binding of soraphen A or an analog thereof to an acetyl CoA carboxylase biotin carboxylase domain (e.g., the biotin carboxylase domain of yeast ACC, human ACC1, or human ACC2, e.g., as determined by in vitro competitive binding assays in accordance with known techniques).

The compounds identified by the methods of the invention, when bound, come within seven angstroms of residues Lys73, Arg76, Ser77, Glu392, and Glu 477 of yeast ACC, or a corresponding biotin carboxylase binding domain of another acetyl CoA carboxylase such as Ustilago mayadis carboxylase, Phytophthora infestans carboxylase, Magnaporthe grisea carboxylase, human ACC1, and human ACC2 (e.g., as determined by molecular modeling or computer-based techniques utilizing the molecular information disclosed herein carried out in accordance with known techniques).

Salts

The compounds described herein and, optionally, all their isomers may be obtained in the form of their salts. Because some of the compounds have a basic center they can, for example, form acid addition salts. Said acid addition salts are, for example, formed with mineral acids, typically sulfric acid, a phosphoric acid or a hydrogen halide, with organic carboxylic acids, typically acetic acid, oxalic acid, malonic acid, maleic acid, fumaric acid or phthalic acid, with hydroxycarboxylic acids, typically ascorbic acid, lactic acid, malic acid, tartaric acid or citric acid, or with benzoic acid, or with organic sulfonic acids, typically methanesulfonic acid or p-toluenesulfonic acid. Together with at least one acidic group, the compounds can also form salts with bases. Suitable salts with bases are, for example, metal salts, typically alkali metal salts; or alkaline earth metal salts, e.g. sodium salts, potassium salts or magnesium salts, or salts with ammonia or an organic amine, e.g. morpholine, piperidine, pyrrolidine, a mono-, di- or trialkylamine, typically ethylamine, diethylamine, triethylamine or dimethylpropylamine, or a mono-, di- or trihydroxyalkylamine, typically mono-, di- or triethanolamine. Where appropriate, the formation of corresponding internal salts is also possible. Within the scope of this invention, agrochemical or pharmaceutically acceptable salts are preferred.

Agrochemical Compositions and Use

Active compounds of the present invention can be used to prepare agrochemical compositions and used to control fungi in like manner as other antifungal compounds. See, e.g., U.S. Pat. No. 6,617,330; see also U.S. Pat. Nos. 6,616,952; 6,569,875; 6,541,500, and 6,506,794. Active compounds described herein can be used for protecting plants against diseases that are caused by fungi. For the purposes herein, oomycetes shall be considered fungi. The active compounds can be used in the agricultural sector and related fields as active ingredients for controlling plant pests. The active compounds can be used to inhibit or destroy the pests that occur on plants or parts of plants (fruit, blossoms, leaves, stems, tubers, roots) of different crops of useful plants, optionally while at the same time protecting also those parts of the plants that grow later e.g. from phytopathogenic micro-organisms.

Active compounds may be used as dressing agents for the treatment of plant propagation material, in particular of seeds (fruit, tubers, grains) and plant cuttings (e.g. rice), for the protection against fungal infections as well as against phytopathogenic fungi occurring in the soil.

The active compounds may be used, for example, against the phytopathogenic fungi of the following classes: Fungi imperfecti (e.g. Botrytis, Pyricularia, Heiminthosporium, Fusarium, Septoria, Cercospora and Alternaria) and Basidiomycetes (e.g. Rhizoctonia, Hemileia, Puccinia). Additionally, they may also be used against the Ascomycetes classes (e.g. Venturia and Erysiphe, Podosphaera, Monilinia, Uncinula) and of the Oomycetes classes (e.g. Phytophthora, Pythium, Plasmopara).

Target crops to be protected with active compounds and compositions of the invention typically comprise the following species of plants: cereal (wheat, barley, rye, oat, rice, maize, sorghum and related species); beet (sugar beet and fodder beet); pomes, drupes and soft fruit (apples, pears, plums, peaches, almonds, cherries, strawberries, raspberries and blackberries); leguminous plants (beans, lentils, peas, soybeans); oil plants (rape, mustard, poppy, olives, sunflowers, coconut, castor oil plants, cocoa beans, groundnuts); cucumber plants (pumpkins, cucumbers, melons); fiber plants (cotton, flax, hemp, jute); citrus fruit (oranges, lemons, grapefruit, mandarins); vegetables (spinach, lettuce, asparagus, cabbages, carrots, onions, tomatoes, potatoes, paprika); lauraceae (avocado, cinnamon, camphor) or plants such as tobacco, nuts, coffee, eggplants, sugar cane, tea, pepper, vines, hops, bananas, turf and natural rubber plants, as well as ornamentals (flowers, shrubs, broad-leafed trees and evergreens, such as conifers). This list does not represent any limitation.

The active compounds can be used in the form of compositions and can be applied to the crop area or plant to be treated, simultaneously or in succession with further compounds. These further compounds can be e.g. fertilizers or micronutrient donors or other preparations which influence the growth of plants. They can also be selective herbicides as well as insecticides, fungicides, bactericides, nematicides, molluscicides, plant growth regulators, plant activators or mixtures of several of these preparations, if desired together with further carriers, surfactants or application promoting adjuvants customarily employed in the art of formulation.

The active compounds can be mixed with other fungicides, resulting in some cases in unexpected synergistic activities.

Mixing components which are particularly preferred are azoles such as azaconazole, bitertanol, propiconazole, difenoconazole, diniconazole, cyproconazole, epoxiconazole, fluquinconazole, flusilazole, flutriafol, hexaconazole, imazalil, imibenconazole, ipconazole, tebuconazole, tetraconazole, fenbuconazole, metconazole, myclobutanil, perfurazoate, penconazole, bromuconazole, pyrifenox, prochloraz, triadimefon, triadimenol, triflumizole or triticonazole; pyrimidinyl carbinoles such as ancymidol, fenarimol or nuarimol; 2-amino-pyrimidine such as bupirimate, dimethirimol or ethirimol; morpholines such as dodemorph, fenpropidin, fenpropimorph, spiroxamin or tridemorph; anilinopyrimidines such as cyprodinil, pyrimethanil or mepanipyrim; pyrroles such as fenpiclonil or fludioxonil; phenylamides such as benalaxyl, furalaxyl, metalaxyl, R-metalaxyl, ofurace or oxadixyl; benzimidazoles such as benomyl, carbendazim, debacarb, fuberidazole or thiabendazole; dicarboximides such as chlozolinate, dichlozoline, iprodine, myclozoline, procymidone or vinclozolin; carboxamides such as carboxin, fenfuram, flutolanil, mepronil, oxycarboxin or thifluzamide; guanidines such as guazatine, dodine or iminoctadine; strobilurines such as azoxystrobin, kresoxim-methyl, metominostrobin, SSF-129, methyl 2[(2-trifluoromethyl)-pyrid-6-yloxymethyl]-3-methoxy-acrylate or 2-[{.alpha.[(.alpha.-methyl-3-trifluoromethyl-benzyl)imino]-oxy}-o-tolyl]-glyoxylic acid-methylester-O-methyloxime (trifloxystrobin); dithiocarbamates such as ferbam, mancozeb, maneb, metiram, propineb, thiram, zineb or ziram; N-halomethylthio-dicarboximides such as captafol, captan, dichlofluanid, fluoromide, folpet or tolyfluanid; copper compounds such as Bordeaux mixture, copper hydroxide, copper oxychloride, copper sulfate, cuprous oxide, mancopper or oxine-copper; nitrophenol derivatives such as dinocap or nitrothal-isopropyl; organo phosphorous derivatives such as edifenphos, iprobenphos, isoprothiolane, phosdiphen, pyrazophos or toclofos-methyl; and other compounds of diverse structures such as acibenzolar-S-methyl, harpin, anilazine, blasticidin-S, chinomethionat, chloroneb, chlorothalonil, cymoxanil, dichlone, diclomezine, dicloran, diethofencarb, dimethomorph, dithianon, etridiazole, famoxadone, fenamidone, fentin, ferimzone, fluazinam, flusulfamide, fenhexamid, fosetyl-aluminium, hymexazol, kasugamycin, methasulfocarb, pencycuron, phthalide, polyoxins, probenazole, propamocarb, pyroquilon, quinoxyfen, quintozene, sulfur, triazoxide, tricyclazole, triforine, validamycin, (S)-5-methyl-2-methylthio-5-phenyl-3-phenylamino-3,5-di-hydroimidazol-4-one (RPA 407213), 3,5-dichloro-N-(3 -chloro-1-ethyl-1-methyl-2-oxopropyl)-4-methylbenzamide (RH-7281), N-allyl-4,5-dimethyl-2-trimethylsilylthiophene-3-carboxamide (MON 65500), 4-chloro-4-cyano-N,N-dimethyl-5-p-tolylimidazole-1-sulfon-amide (IKF-916), N-(1 -cyano-1,2-dimethylpropyl)-2-(2,4-dichlorophenoxy)-propionamide (AC 382042) or iprovalicarb (SZX 722).

Suitable carriers and adjuvants can be solid or liquid and are substances useful in formulation technology, e.g. natural or regenerated mineral substances, solvents, dispersants, wetting agents, tackifiers, thickeners, binders or fertilizers.

A preferred method of applying an active compound of the invention, or an agrochemical composition which contains at least one of said compounds, is foliar application. The frequency of application and the rate of application will depend on the risk of infestation by the corresponding pathogen. However, the active compounds can also penetrate the plant through the roots via the soil (systemic action) by drenching the locus of the plant with a liquid formulation, or by applying the compounds in solid form to the soil, e.g. in granular form (soil application). In crops of water such as rice, such granulates can be applied to the flooded rice field. The active compounds may also be applied to seeds (coating) by impregnating the seeds or tubers either with a liquid formulation of the fungicide or coating them with a solid formulation.

The term locus as used herein is intended to embrace the fields on which the treated crop plants are growing, or where the seeds of cultivated plants are sown, or the place where the seed will be placed into the soil. The term seed is intended to embrace plant propagating material such as cuttings, seedlings, seeds, and germinated or soaked seeds.

The active compounds are used in unmodified form or, preferably, together with the adjuvants conventionally employed in the art of formulation. To this end they are conveniently formulated in known manner to emulsifiable concentrates, coatable pastes, directly sprayable or dilutable solutions, dilute emulsions, wettable powders, soluble powders, dusts, granulates, and also encapsulations e.g. in polymeric substances. As with the type of the compositions, the methods of application, such as spraying, atomizing, dusting, scattering, coating or pouring, are chosen in accordance with the intended objectives and the prevailing circumstances.

Advantageous rates of application are normally from 5 g to 2 kg of active ingredient (a.i.) per hectare (ha), preferably from 10 g to 1 kg a.i./ha, most preferably from 20 g to 600 g a.i./ha. When used as seed drenching agent, convenient dosages are from 10 mg to 1 g of active substance per kg of seeds.

The formulation, i.e. the compositions containing the compound of formula I and, if desired, a solid or liquid adjuvant, are prepared in known manner, typically by intimately mixing and/or grinding the compound with extenders, e.g. solvents, solid carriers and, optionally, surface active compounds (surfactants).

Suitable carriers and adjuvants may be solid or liquid and correspond to the substances ordinarily employed in formulation technology, such as, e.g. natural or regenerated mineral substances, solvents, dispersants, wetting agents, tackifiers, thickeners binding agents or fertilizers. Such carriers are for example described in WO 97/33890.

Further surfactants customarily employed in the art of formulation are known to the expert or can be found in the relevant literature.

The agrochemical formulations will usually contain from 0.1 to 99% by weight, preferably from 0.1 to 95% by weight, of the compound of formula I, 99.9 to 1% by weight, preferably 99.8 to 5% by weight, of a solid or liquid adjuvant, and from 0 to 25% by weight, preferably from 0.1 to 25% by weight, of a surfactant.

Whereas it is preferred to formulate commercial products as concentrates, the end user will normally use dilute formulations.

The compositions may also contain further adjuvants such as stabilizers, antifoams, viscosity regulators, binders or tackifiers as well as fertilizers, micronutrient donors or other formulations for obtaining special effects.

Technical Materials

The compounds and combinations of the present invention may also be used in the area of controlling fungal infection (particularly by mold and mildew) of technical materials, including protecting technical material against attack of fungi and reducing or eradicating fungal infection of technical materials after such infection has occurred. Technical materials include but are not limited to organic and inorganic materials wood, paper, leather, natural and synthetic fibers, composites thereof such as particle board, plywood, wall-board and the like, woven and non-woven fabrics, construction surfaces and materials, cooling and heating system surfaces and materials, ventilation and air conditioning system surfaces and materials, and the like. The compounds and combinations according the present invention can be applied to such materials or surfaces in an amount effective to inhibit or prevent disadvantageous effects such as decay, discoloration or mold in like manner as described above. Structures and dwellings constructed using or incorporating technical materials in which such compounds or combinations have been applied are likewise protected against attack by fungi.

5. Pharmaceutical Uses

In addition to the foregoing, active compounds of the present invention can be used in the treatment of fungal infections of human and animal subjects (including but not limited to horses, cattle, sheep, dogs, cats, etc.) for medical and veterinary purposes. Examples of such infections include but are not limited to ailments such as Onychomycosis, sporotichosis, hoof rot, jungle rot, Pseudallescheria boydii, scopulariopsis or athletes foot, sometimes generally referred to as “white-line” disease, as well as fungal infections in immunocomprised patients such as AIDS patients and transplant patients. Thus, fungal infections may be of skin or of keratinaceous material such as hair, hooves, or nails, as well as systemic infections such as those caused by Candida spp., Cryptococcus neoformans, and Aspergillus spp., such as as in pulmonary aspergillosis and Pneumocystis carinii pneumonia. Active compounds as described herein may be combined with a pharmaceutically acceptable carrier and administered or applied to such subjects or infections (e.g., topically, parenterally) in an amount effective to treat the infection in accordance with known techniques, as (for example) described in U.S. Pat. Nos. 6,680,073; 6,673,842; 6,664,292; 6,613,738; 6,423,519; 6,413,444; 6,403,063; and 6,042,845; the disclosures of which applicants specifically intend be incoroporated by reference herein in their entirety.

In addition to the foregoing, the compounds may be used for the treatment of obesity, metabolic syndrome or insulin resistance, e.g., type II or adult-onset diabetes, in human or animal subjects. “Pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. “Pharmaceutically-acceptable carrier” as used herein means a pharmaceutically-acceptable material, composition or vehicle, such as a liquid or solid filler, diluent, excipient, solvent or encapsulating material, involved in carrying or transporting the subject peptidomimetic agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the active ingredient which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association a compound of the present invention with the carrier and, optionally, one or more accessory ingredients. In general, the formulations are prepared by uniformly and intimately bringing into association a peptide or peptidomimetic of the present invention with liquid carriers, or finely divided solid carriers, or both, and then, if necessary, shaping the product.

The ointments, pastes, creams and gels may contain, in addition to the active ingredient, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Formulations suitable for oral administration may be presented in discrete units, such as capsules, cachets, lozenges, or tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the active compound and a suitable carrier (which may contain one or more accessory ingredients as noted above). In general, the formulations of the invention are prepared by uniformly and intimately admixing the active compound with a liquid or finely divided solid carrier, or both, and then, if necessary, shaping the resulting mixture. For example, a tablet may be prepared by compressing or molding a powder or granules containing the active compound, optionally with one or more accessory ingredients. Compressed tablets may be prepared by compressing, in a suitable machine, the compound in a free-flowing form, such as a powder or granules optionally mixed with a binder, lubricant, inert diluent, and/or surface active/dispersing agent(s). Molded tablets may be made by molding, in a suitable machine, the powdered compound moistened with an inert liquid binder.

Pharmaceutical compositions of this invention suitable for parenteral administration comprise one or more active compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. These compositions may also contain adjuvants such as preservatives, wetting agents, emulsifying agents and dispersing agents. Prevention of the action of microorganisms may be ensured by the inclusion of various antibacterial and other antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

The preparations of the present invention may be given by any suitable means of administration including orally, parenterally, topically, transdermally, rectally, etc. They are of course given by forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Topical or parenteral administration is preferred. “Parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal and intrasternal injection and infusion.

Actual dosage levels of the active ingredients in the pharmaceutical compositions of this invention may be varied so as to obtain an amount of the active ingredient which is effective to achieve the desired therapeutic response, e.g., antimycotic activity, for a particular patient, composition, and mode of administration, without being toxic to the patient. The selected dosage level will depend upon a variety of factors including the activity of the particular active compound employed, the route of administration, the time of administration, the rate of excretion of the particular active compound being employed, the duration of the treatment, other drugs, compounds and/or materials used in combination with the particular inhibitor employed, the age, sex, weight, condition, general health and prior medical history of the patient being treated, and like factors well known in the medical arts. A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved. As a general proposition, a dosage from about 0.01 or 0.1 to about 50, 100 or 200 mg/kg will have therapeutic efficacy, with all weights being calculated based upon the weight of the active compound, including the cases where a salt is employed.

The following examples are representative of techniques employed by the inventors in carrying out aspects of the present invention. It should be appreciated that while these techniques are exemplary for the practice of the invention, those of skill in the art, in light of the present disclosure, will recognize that numerous modifications can be made without departing from the spirit and intended scope of the invention.

### EXAMPLE 1

To reveal the molecular mechanism for the potent inhibitory activity of this natural product against the eukaryotic ACCs, we have determined the crystal structure of the yeast BC domain in complex with soraphen A at 1.8 Å resolution. The structure reveals extensive interactions between soraphen and the BC domain, explaining its strong affinity. Large structural differences between the eukaryotic and bacterial BC in the soraphen binding site precludes the binding of soraphen to the bacterial enzymes. Unexpectedly, our structures suggest soraphen may have a novel mechanism of inhibiting the BC domain. It may bind in the dimer interface, thereby disrupting the oligomerization of this domain, which is crucial for its catalytic activity. The structural observation is supported by our native gel electrophoresis experiments. We have developed a fluorescence-based binding assay, which allowed us to characterize the effects of single-site mutations in the soraphen binding site on inhibitor sensitivity.

### A. Experimental Procedures

Protein Expression and Purification

The expression and purification of the yeast BC domain followed the protocols that we have described for the Ustilago BC domain (Weatherly et al., supra (2004). Residues 2-581 of yeast ACC were sub-cloned into the pET28a vector (Novagen) to create pCS16 and over-expressed in E. coli BL21(DE3) Rosetta cells (Novagen) at 20° C. The soluble protein was purified by nickel agarose, anion exchange and gel-filtration chromatography. The purified BC domain was concentrated to 60 mg/ml in a buffer containing 100 mM Tris (pH 8.5), 100 mM NaCl, 5% (v/v) glycerol and 5 mM DTT. The recombinant protein contains an N-terminal hexa-histidine tag, together with about 30 other residues from the expression vector. These residues were not removed for crystallization.

The selenomethionyl protein was produced in B834(DE3) cells (Novagen), grown in defined LeMaster media supplemented with selenomethionine (Hendrickson, W. A. et al., EMBO J 9, 1665-1672 (1990)), and purified following the same protocol as that for the native protein. The selenomethionyl protein was concentrated to 50 mg/ml in a solution of 100 mM Tris (pH 8.5), 150 mM NaCl, 5% (v/v) glycerol and 8 mM DTT.

Protein Crystallization

Crystals of yeast BC domain in complex with soraphen A were obtained at 22 ° C. by the sitting-drop vapor difflusion method. The protein at 50 mg/ml was incubated with 0.88 mM soraphen A (protein:inhibitor molar ratio of 1:1.2) at 4° C. for 1 hour prior to crystallization. The reservoir solution contains 100 mM Bis-Tris (pH 6.0), 26% (w/v) PEG3350, 200 mM NaCl and 400 mM MgCl₂. The crystals grew to full size in about 12-18 days, and micro-seeding was necessary to obtain crystals of diffraction quality. The crystals were cryo-protected by transferring to the reservoir solution supplemented with 9% glycerol and flash-frozen in liquid propane for data collection at 100K. They belong to space group P2₁, with cell parameters of a=63.83 Å, b=96.52 Å, c=139.95 Å, and β=96.82°. There are three copies of the BC:soraphen complex in the asymmetric unit.

Crystals of the selenomethionyl protein in complex with soraphen A were grown with the sitting-drop vapor diffusion method at 22° C. The reservoir solution contained 100 mM Bis-Tris (pH 5.8), 26% (w/v) PEG3350, 100 mM NaCl, 200 mM MgCl₂, 8% glycerol and 2 mM DTT. Micro-seeding from the native crystals was essential. The crystals are isomorphous to those of the native protein.

Crystals of the free enzyme of yeast BC domain was obtained by sitting-drop vapor diffusion method at 4° C. The reservoir solution contained 100 mM Bis-Tris propane (pH 6.0), 23 % (w/v) PEG3350, 200 mM NaCl, 400 mM MgCl₂, and 5% glycerol. The crystals belong to space group P6₂, with cell parameters of a=b=101.74 Å, and c=145.83 Å. There is one molecule of the BC domain in the asymmetric unit. Crystallographic analysis suggests that the crystal is almost perfectly merohedrally twinned, as the diffraction data display 6/mmm symmetry.

Structure Determination

X-ray diffraction data were collected at the X4A beamline of the National Synchrotron Light Source (NSLS). The diffraction images were processed with the HKL package (Otwinowski, Z. et al., Method Enzymol 276, 307-326 (1997)). A selenomethionyl multi-wavelength anomalous diffraction (MAD) data set to 2.9 Å resolution and a native data set to 1.8 Å resolution were collected. The MAD data were loaded into the program Solve (Terwilliger, T. C. et al., Acta Cryst D55, 849-861 (1999)), which located the Se sites, phased the reflections, and built partial models for three molecules of the BC domain.

The non-crystallographic symmetry (NCS) parameters were determined based on the partial models, and the reflection phases were transferred to the native data set. The phase information was extended to 1.8 Å resolution by NCS averaging with the program DM (The CCP4 suite: programs for protein crystallography. Acta Cryst D50, 760-763 (1994)), and Solve was able to automatically build in 60% of the residues into this map. Additional residues were built manually with the program O (Jones, T. A. et al., Acta Cryst A47, 110-119 (1991)). The structure refinement was carried out with the program CNS (Brunger, A. T. et al., Acta Cryst D54, 905-921 (1998)). Residues 248 and 333 are modeled as cis prolines, and their equivalents in E. coli BC are also in the cis conformation (Waldrop, G. L. et al., Biochem 33, 10249-10256 (1994)). The crystallographic information is summarized in Table 2.

The structure of the free enzyme of yeast BC was determined by the molecular replacement method with the program COMO (Jogl, G. et al., Acta Cryst D57, 1127-1134 (2001)). The diffraction data on this crystal had apparent P6/mmm symmetry, and the twinning fraction was estimated to be 0.5. Based on the atomic model, the diffraction data set was de-twinned, using standard procedures in the CNS program (Brunger et al., supra (1998)), and structure refinement was performed against this modified data set.

Mutagenesis and Binding Assays

The mutants were designed based on the structural information and made with the QuikChange kit (Stratagene). The mutants were sequenced, expressed in E. coli, and purified following the same protocol as that for the wild-type BC domain. The affinity of soraphen for the mutants were assessed using a radioactive binding assay (Weatherly et al., supra (2004)).

We have developed a fluorescence-based binding assay using our structural information, which monitored the increase in Trp emission upon soraphen binding. The binding buffer initially contained 100 mM Tris (pH 8.0), 100 mM NaCl, and 50 nM wild-type or mutant enzyme, and increasing concentrations of soraphen A was titrated into the solution. The observed binding curve is fitted using conventional methods or the tight-binding model where appropriate.

### B. Results and Discussion

Structure Determination

The crystal structure of the BC domain of yeast ACC in complex with soraphen A was determined at 2.9 Å resolution by the seleno-methionyl multi-wavelength anomalous diffraction (MAD) technique (Hendrickson, W. A., Science 254, 51 -58 (1991)). These seleno-methionyl crystals actually diffracted to much higher resolution at the beginning of the experiment, but they suffered serious radiation damage during the data collection. Good quality diffraction lasted only about 5 hours in the X-ray beam, and the exposure time per frame was drastically reduced in order to collect a complete three-wavelength MAD data set in this time. This restricted the diffraction limit of the data set to 2.9 Å resolution.

The positions of the Se atoms and the phases of the reflections were determined from the MAD data with the program Solve (Terwilliger and Berendzen, supra (1999)), and the non-crystallographic symmetry (NCS) relationships among the three molecules of the BC domain in the crystallographic asymmetric unit were determined based on the resulting atomic model. The phase information was transferred to a data set to 1.8 Å resolution collected on a native crystal (Table 1), and NCS averaging, with the program DM (CCP4, 1994), was used to improve and extend the phases. The electron density map at 1.8 Å resolution was of excellent quality, and most of the atomic model was built automatically (Terwilliger and Berendzen, supra (1999)).

Interestingly, several attempts at solving the structure using the single-wavelength anomalous diffraction (SAD) method were not successful, as it was not possible to locate the Se atoms based on the SAD data. After the structure was solved by the MAD method, the Se atoms could be positioned with anomalous difference electron density maps using the SAD data. However, these Se sites appeared to have weaker peak heights in the difference maps, which might explain the difficulty in locating them from Patterson or direct methods.

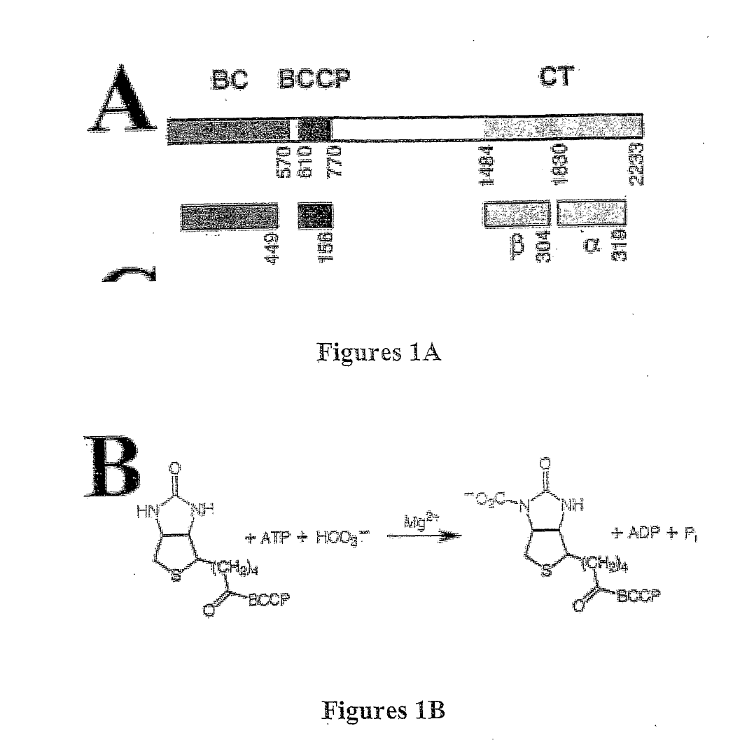

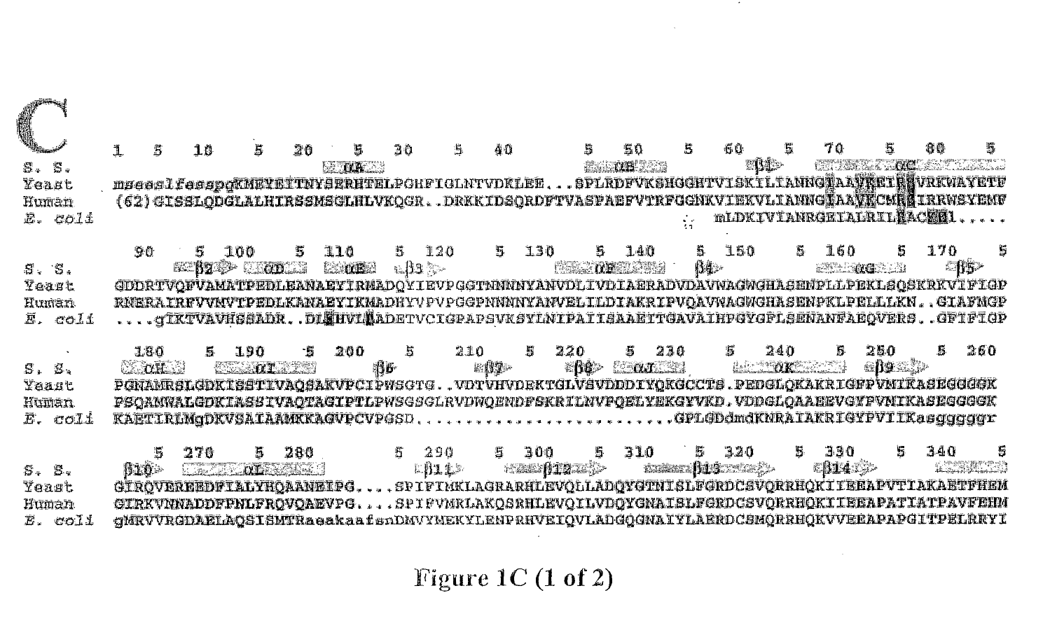

The BC domain of yeast ACC shares 35% amino acid sequence identity with the BC subunit of E. coli (FIG. 1C), for which crystal structures are available (Thoden et al., supra (2000); Waldrop et al., supra (1994)). Attempts at solving the structure of the yeast BC domain by molecular replacement were not successful either, which is likely due to the large structural differences between the two enzymes (see below).

The three BC domain molecules in the asymmetric unit do not form dimeric or trimeric association in the crystal, consistent with our light scattering studies showing that the BC domain is monomeric in solution. Two of the BC domains have essentially the same conformation, with rms distance of 0.4 Å between their equivalent Cα atoms. The third BC domain show recognizable conformational differences for several loops on the surface of the enzyme, but these are not in the soraphen binding site. Soraphen A has the same binding mode in the three copies of the BC-soraphen complexes in the asymmetric unit.

The Overall Structure

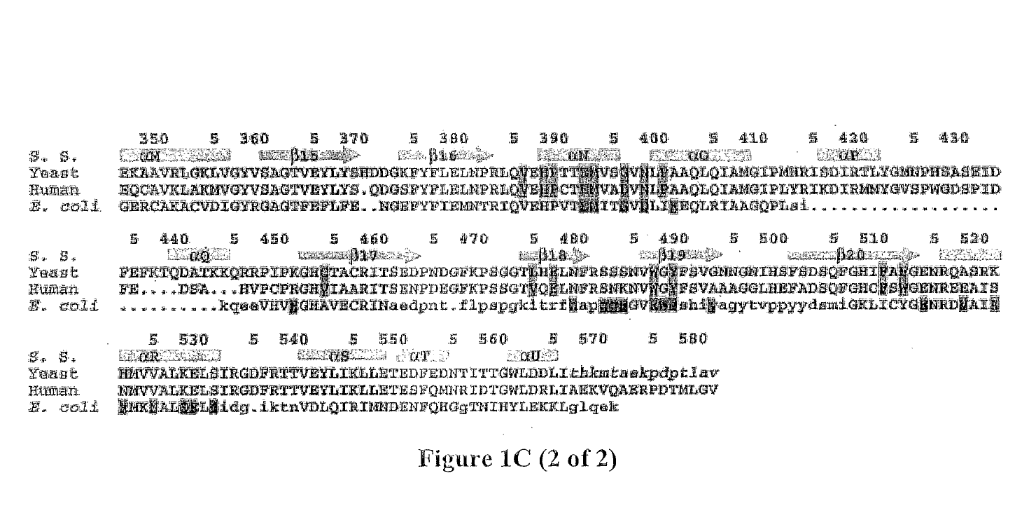

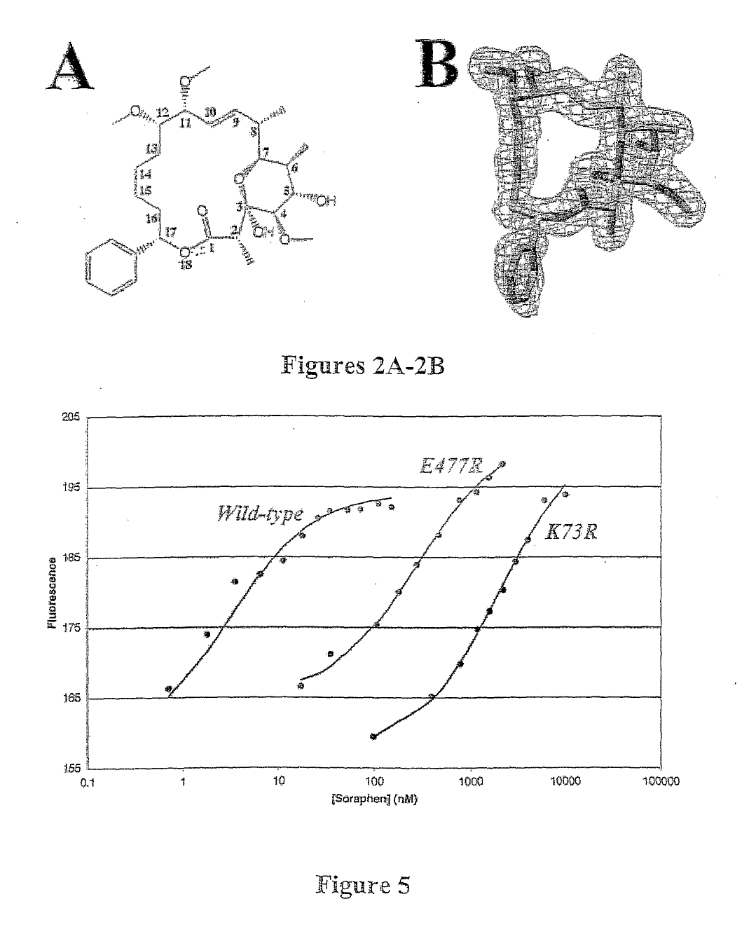

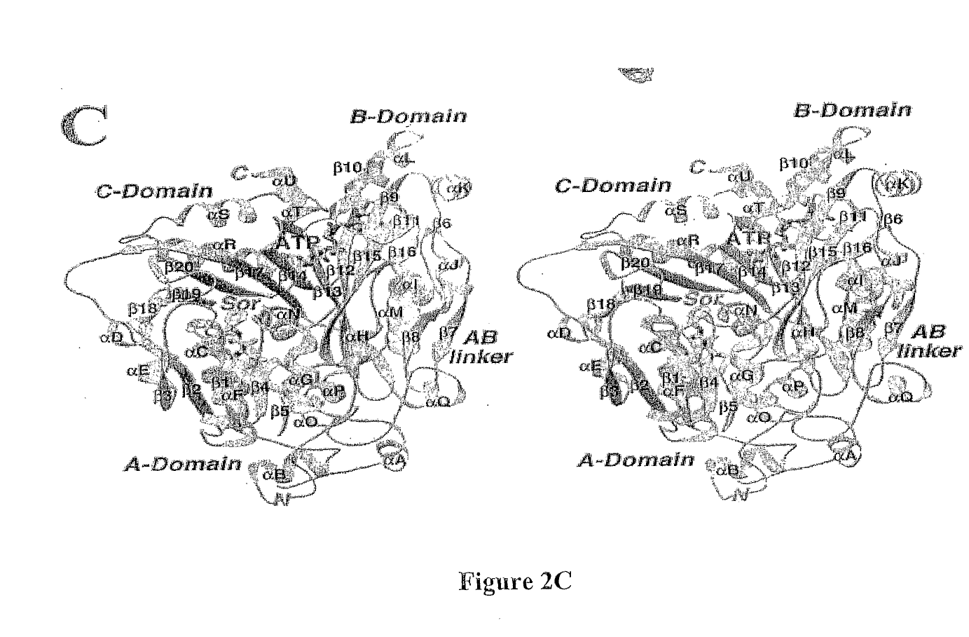

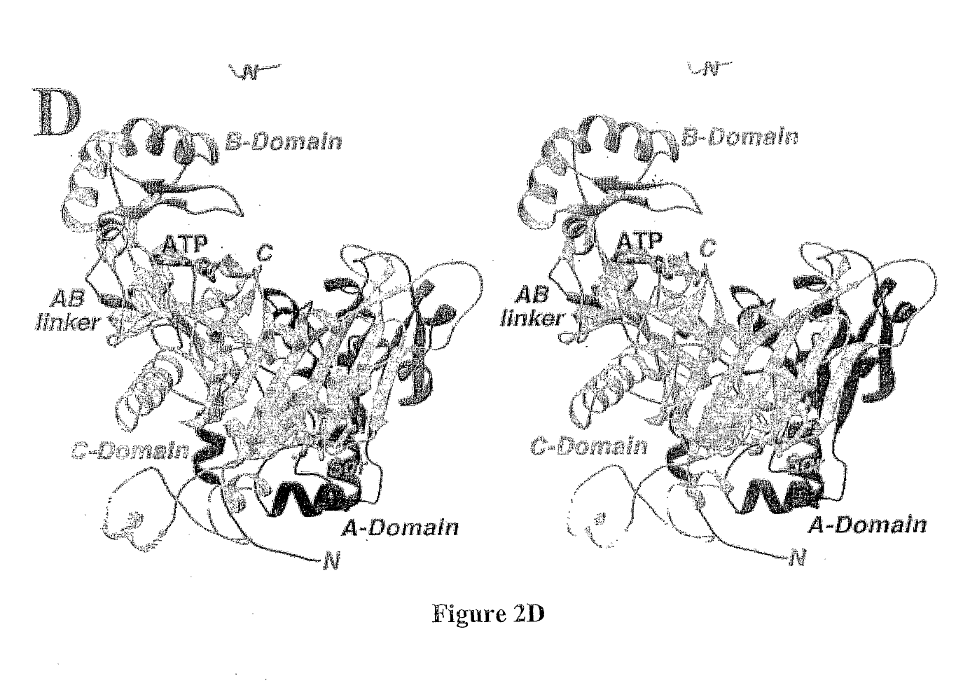

The crystal structure of the BC domain of yeast ACC in complex with soraphen A has been determined at 1.8 Å resolution. The current atomic model has an R factor of 19.5% (Table 2). The bound conformation of soraphen A is clearly defined by the crystallographic analysis (FIG. 2B). The majority of the residues (91.6%) are in the most favored region, while none of the residues are in the disallowed region, of the Ramachandran plot (data not shown). The atomic coordinates of various crystal structures of the invention are shown in tables 4-6 below.

The structure of the yeast BC domain contains 20 β-strands (named β1 through β20) and 21 α-helices (αA through αU) (FIG. 2C). The overall structure of the BC domain has the ATP-grasp fold (Artymiuk, P. J. et al., Nature Struct Biol 3, 128-132 (1996); Galperin, M. Y., and Koonin, E. V., Protein Sci 6, 2639-2643 (1997)), and consists of three sub-domains (FIG. 2D) (Thoden et al., supra (2000); Waldrop et al., supra (1994)). The A-domain covers residues 1-175 (strands β1-β5, helices αA-αG) and has the Rossmann-fold, with a central five-stranded fully parallel β-sheet. The B-domain (residues 234-293, with β9-β11, αK and αL) contains a three-stranded anti-parallel β-sheet with two helices (FIG. 2D). A small strand (β6) from the AB linker (residues 176-233, with β6-β8, αH-αJ) extends this β-sheet to four strands (FIG. 2C). The C-domain (residues 294-566) contains a nine-stranded anti-parallel β-sheet (β12 through β20), with helices (αM-αU) on both sides (FIG. 2C).

The B-domain of E. coli BC subunit undergoes a large conformational change upon ATP binding (Thoden et al., supra (2000)), and assumes a closed conformation. The B-domain of yeast BC in the soraphen complex is mostly in the closed conformation, even though ATP is not bound in the active site (FIG. 2C).

The Binding Mode of Soraphen

Our structure demonstrates that soraphen A is an allosteric inhibitor of the BC domain, as it is located 25 Å away from the putative position of the ATP molecule in the active site, on the opposite surface of the enzyme (FIG. 2D). The A-domain, C-domain, and AB-linker form a cylindrical structure, with the ATP and soraphen molecules located on opposite ends of this cylinder, while the B-domain is a lid on the cylinder (FIG. 2D). The structural observation is consistent with kinetic data showing that soraphen A is generally noncompetitive with respect to the substrates of ACC (Behrbohm, supra (1996)).

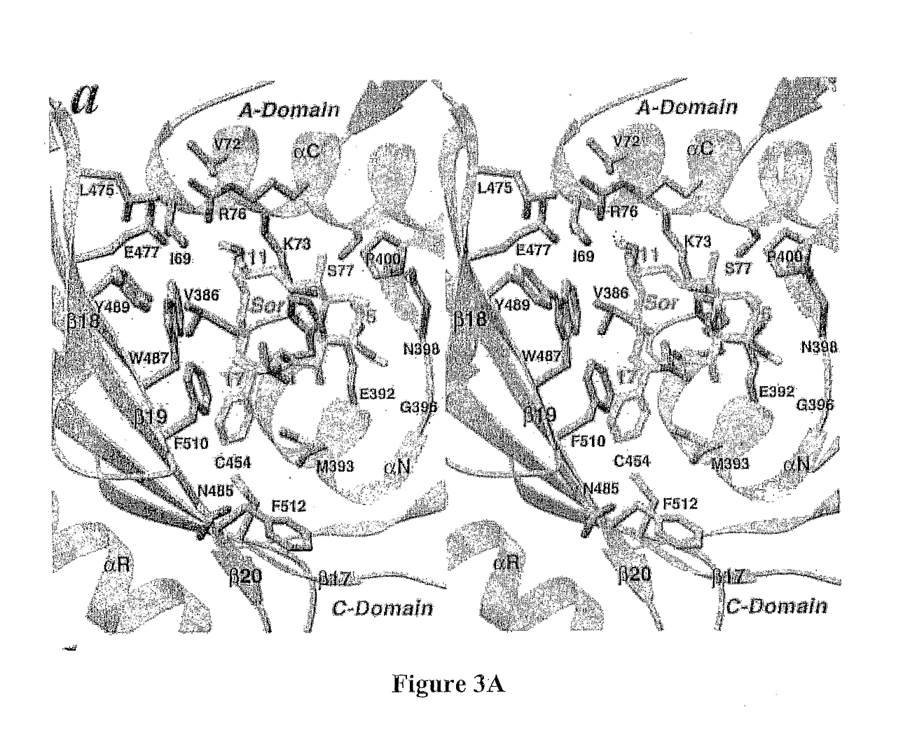

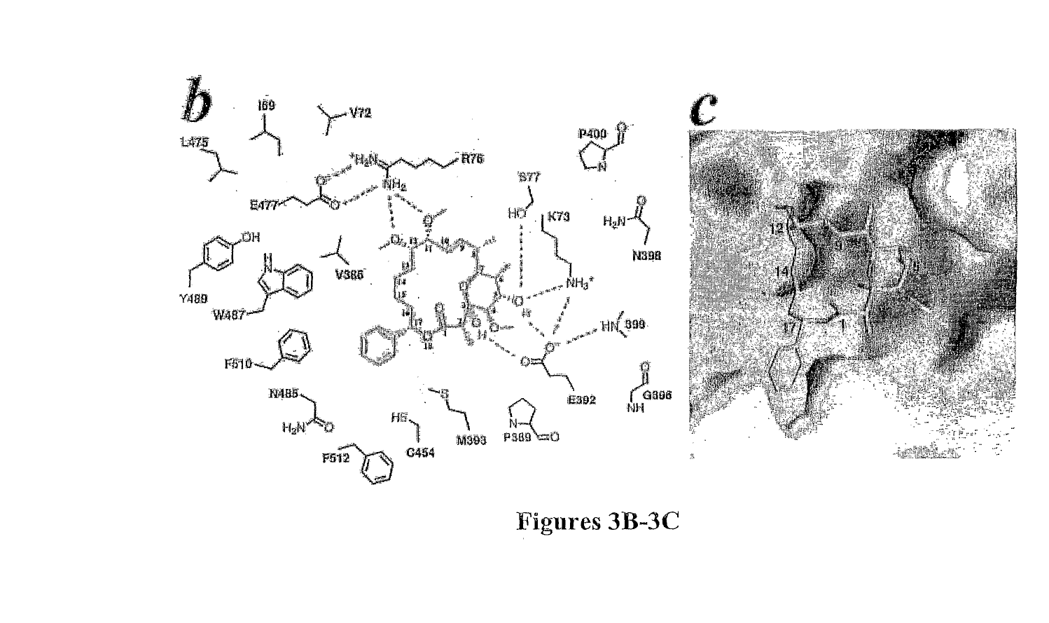

There are extensive interactions between soraphen A and the BC domain (FIG. 3A), explaining the nanomolar binding affinity of this natural product. In addition, most of the residues that are involved in binding soraphen A are highly conserved among the BC domains of eukaryotic ACCs (FIG. 1C), consistent with the potent activity of this compound against all of them. For example, the K_{d} of soraphen for the BC domains of human ACC1 and ACC2 is ˜1 nM (unpublished results). The potent activity and the strong sequence conservation between the yeast and human BC domains suggest that soraphen should have the same binding mode to the human BC domains.

Soraphen A is bound at the interface between the A-domain and C-domain (FIG. 3A), having interactions with residues in strands β17-β20 and helices αN, αO in the C-domain, as well as several critical residues from helix αC in the A-domain (FIG. 3B). One wall of the binding site is formed by strands β17-β20 in the second half of the C-domain (FIG. 3A). From the αC helix in the A-domain, residues Lys73 and Arg76, in ion-pair interactions with Glu392 (αN) and Glu477 (β18) in the C-domain, respectively, mediate the binding of soraphen A as well as the interactions between the two domains (FIG. 3A). The oxygens of the methoxy groups on C11 and C12 of soraphen are hydrogen-bonded to the side chain of Arg76 (αC) (FIG. 3B). In addition, Ser77 in helix αC is in direct contact with soraphen A, hydrogen-bonded to its C5 hydroxyl group (FIG. 3B).

The bound conformation of soraphen A is essentially the same as that of the compound alone (Bedorf et al., supra (1993)), with the exception of a torsional adjustment of the methoxy group on C12. The macrocycle of the compound is placed on the surface of the BC domain (FIG. 3C), and 300 Å² of the surface area of the BC domain are shielded from the solvent in the complex. The four methylene groups (C13 through C16) and the extracyclic phenyl ring of soraphen A are located in a highly hydrophobic environment, and the side chains of Met393 (αN) and Trp487 (β119) make critical contributions to this binding site. The methoxy group on C12 is located in a small pocket on the surface of the enzyme (FIG. 3C). Interestingly, our structure suggests that small, hydrophobic substituents at C13 or C14 might be able to have favorable interactions with a neighboring pocket (FIG. 3C).

The observed binding mode of soraphen A is supported by biochemical observations. Most importantly, it has been found that mutation of Ser77 of yeast ACC to Tyr renders the enzyme insensitive to soraphen A (Vahlensieck et al., supra (1994; 1997)). Based on our structure, this mutation will introduce steric clash between the Tyr side chain and soraphen (FIG. 3A), thereby disallowing the binding of the compound. The K73R mutation has also been found to confer resistance to soraphen A. The structure suggests that this mutation may disrupt the ion-pair with Glu392, which should be detrimental for the binding of the compound as well (FIG. 3A). Our additional studies show that mutation of other residues in this binding site can also disrupt soraphen binding (see below).

The observed binding mode of soraphen A can also explain the structure-activity relationship (SAR) that has been observed for analogs of this natural product. Our structure of the complex shows that the entire macrocycle of soraphen is involved in binding to the BC domain, consistent with the SAR that sub-structures of soraphen do not have anti-fungal activities (Loubinoux, B. et al., J Chem Soc Perkin Trans 1, 521-526 (1995); Loubinoux, B. et al., Tetrahedron 51, 3549-3558 (1995); Loubinoux, B. et al., Helvetica Chimica Acta 78, 122-128 (1995); Loubinoux, B. et al., J Org Chem 60, 953-959 (1995)). Changing the stereochemistry of the phenyl substituent at C17 abolished the activity of the compound, while replacing the phenyl ring with other groups led to a reduction in activity (Schummer, D. et al., Liebigs Ann, 803-816 (1995)). The trans double bond between C9 and C10 does not have specific interactions with the enzyme (FIG. 3A), and it can be reduced (producing soraphen F) with only a moderate loss of activity (Hofle, G. et al., Tetrahedron 51, 3159-3174 (1995)). Interestingly, removing the hydroxyl group on C5 only produces a 5-fold loss of activity (Kiffe, M. et al., Liebigs Ann, 245-252 (1997)), suggesting that the hydrogen-bond to Ser77 may not be crucial for the activity of soraphen A (FIG. 3B).

Molecular Basis For the Specificity of Soraphen

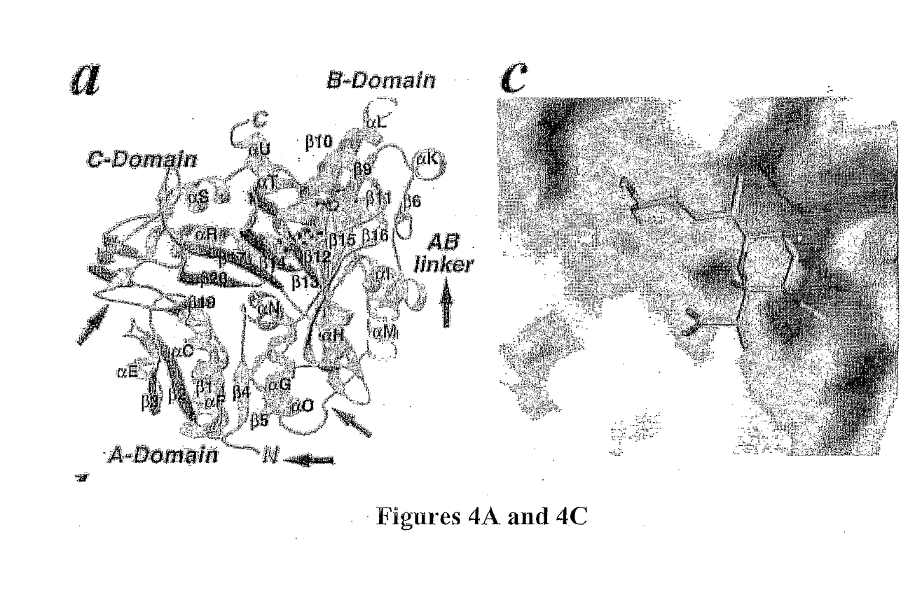

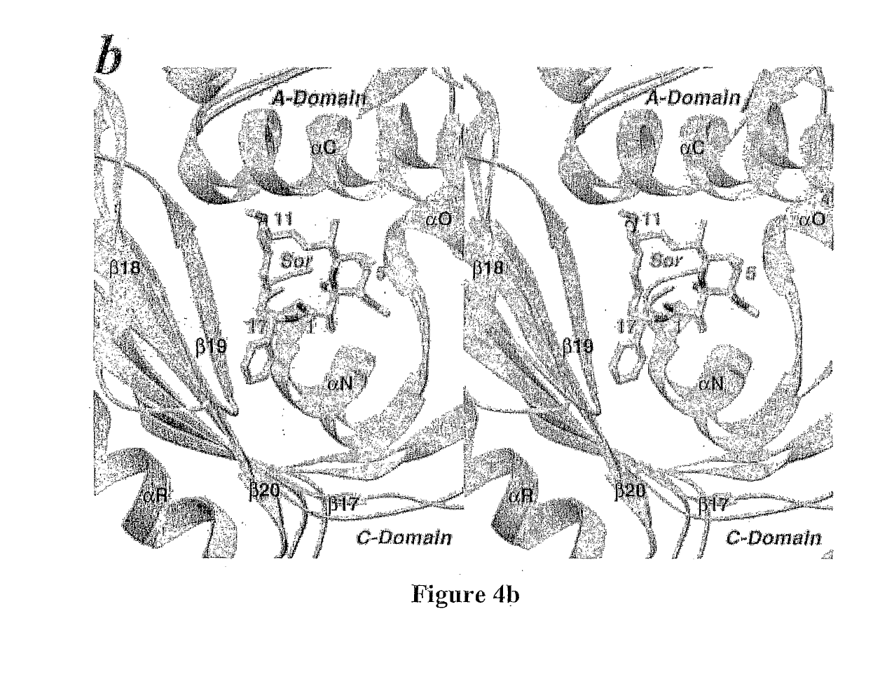

To understand the molecular basis for the specificity of soraphen A for eukaryotic BC domains, we compared the structures of the yeast BC domain and bacterial BC subunit (Thoden et al., supra (2000); Waldrop et al., supra (1994)). Despite sharing 35% amino acid sequence identity, there are significant differences between the two structures (FIGS. 2C, 4A). Only 364 of the 447 Cα atoms of the E. coli BC structure can be superimposed to within 3 Å of the yeast BC structure (FIG. 1C), and the rms distance for these equivalent Cα atoms is 1.6 Å. Compared to the bacterial BC subunit, the eukaryotic BC domain has insertions in the A-domain (αA and αB at the N-terminus), AB linker (β7, β8 and αJ), and C-domain (αP and αQ) (FIG. 4A), explaining its larger size.

The largest structural differences between the eukaryotic and bacterial BC are seen in the second half of the C-domain, which is also the binding site for soraphen. The position of strand β19 in bacterial BC shifts by about 3 Å towards the soraphen molecule, and strand β18 is absent in the E. coli BC structure (FIG. 4B). As a consequence, the molecular surface of bacterial BC subunit is incompatible with soraphen A binding (FIG. 4C), and there is serious steric clash between soraphen and residues in strand β19 of the bacterial BC structure. In addition to these differences in main chain conformations, changes in amino acid side chains in this binding site are also detrimental for soraphen binding to the bacterial BC subunit (see below). Overall, structural and amino acid sequence differences between the bacterial and eukaryotic BC determine the specificity of soraphen for eukaryotic ACCs.

A fluorescence-Based Binding Assay

We next developed a fluorescence-based binding assay using the structural information. Our structures show that Trp487 is mostly exposed to the solvent in the free enzyme, but is buried by soraphen A in the complex (FIG. 3A). This suggests that the fluorescence emission of this residue should be enhanced in the complex, which enabled us to establish the fluorescence binding assay (FIG. 5). There is also a slight blue shift in the fluorescence emission maximum upon soraphen binding. The observed increase in Trp fluorescence as a function of soraphen concentration can be easily fit to a one-site binding model (FIG. 5), confirming that there is a single binding site for soraphen in the BC domain. The binding affinity obtained from this fluorescence assay is generally in good agreement with that based on the radioactive binding assay (Table 3) (Weatherly et al., supra (2004)). Compared to the radioactive assay, the fluorescence assay has the advantage that it can measure affinity between 1 nM to 10 μM, whereas the radioactive assay is limited to K_{d} values below ˜50 nM.

The establishment of this fluorescence binding assay allowed us to further characterize the soraphen binding site. We selected those residues in this region that show differences to their equivalents in the E. coli BC subunit, and introduced these changes to yeast BC domain as single-site mutations. These mutants generally have drastically reduced affinity for soraphen (Table 3), confirming the structural information and suggesting another molecular mechanism for the specificity of soraphen for the BC domains of eukaryotic ACCs. The K73R mutant has a 500-fold loss in affinity for soraphen, such that the K_{d} is now in the micromolar range (Table 3). At the same time, the conservative F5101 mutation has only a minor impact on the affinity for soraphen (Table 3).

Finally, there is little fluorescence change for the W487R mutant in the presence of soraphen (data not shown), confirming that the fluorescence increase observed for the wild-type enzyme and the other mutants is due almost exclusively to the Trp487 residue.

Soraphen Binding Causes Little Conformational Changes in the BC Domain

What is the molecular mechanism for the potent inhibitory activity of soraphen A? One possibility is that soraphen A allosterically interferes with either substrate binding or catalysis in the active site. However, based on our structures and the current biochemical information, this is unlikely to be the case. The noncompetitive nature of inhibition by soraphen already suggests that soraphen does not have an allosteric effect on the active site of the enzyme (Behrbohm, supra (1996)). This is corroborated by our structural studies on the free enzyme of the yeast BC domain.

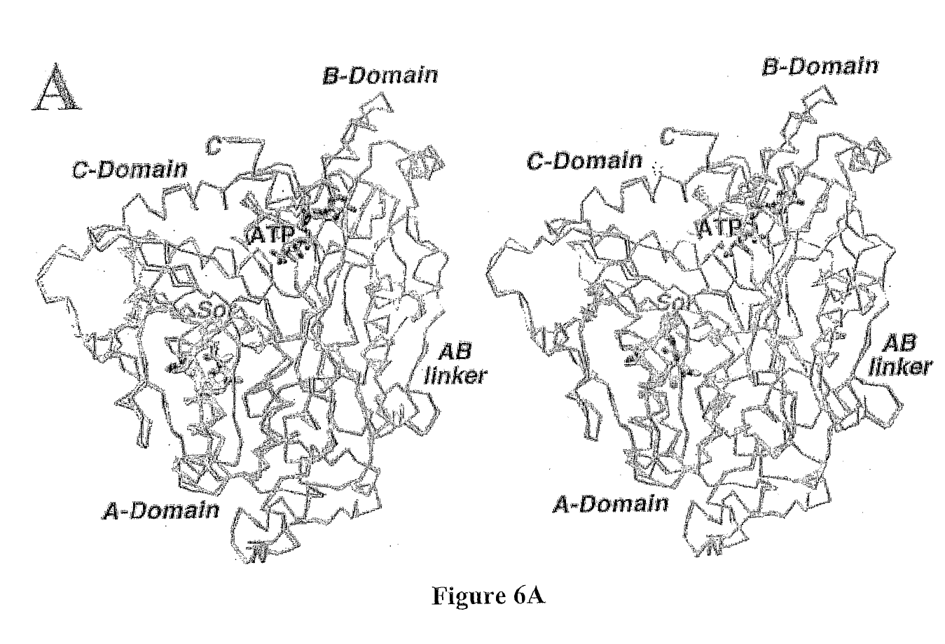

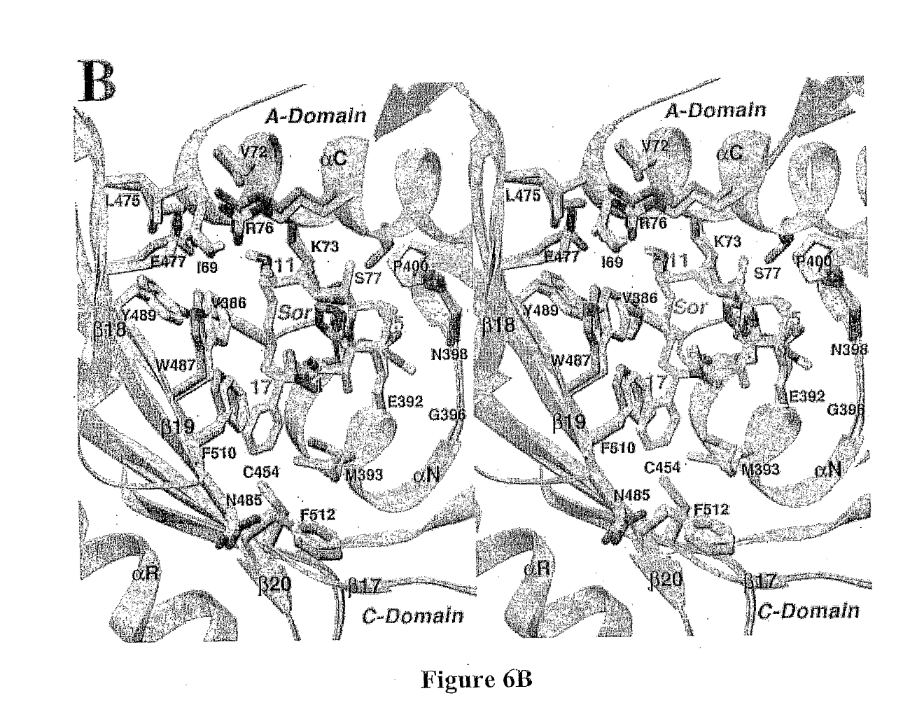

To assess whether there are conformational changes in the BC domain upon soraphen binding, we have determined the crystal structure of the free enzyme of yeast BC domain at 2.5 Å resolution (Table 2). The overall structure of the free enzyme is the same as that of the soraphen complex (FIG. 6A), and the rms distance for all the equivalent Cα atoms of the two structures is 0.6 Å. In addition, there are only small changes in the soraphen binding site (FIG. 6B) and the active site. This suggests that soraphen binding does not induce an overall conformational change in the BC domain, making an allosteric effect for soraphen unlikely.

The structural observation is also supported by our preliminary experiments showing that soraphen A does not interfere with the binding of a fluorescent ATP analog (Mant-ATP) to the active site of yeast BC domain (unpublished data). Interestingly, the B-domain assumes the closed conformation in the yeast BC domain, even in the absence of ATP (FIG. 6A), in sharp contrast to observations from the structure of bacterial BC subunit (Kondo, S. et al., Acta Cryst D60, 486-492 (2004); Thoden et al., supra (2004); Waldrop et al., supra (1994)).

Soraphen May be a Protein-Protein Interaction Inhibitor

Our structural information indicates instead that soraphen A may have a novel mechanism of action. This natural product may function as a protein-protein interaction inhibitor, and abolishes the activity of the BC domain by disrupting its dimerization or oligomerization.

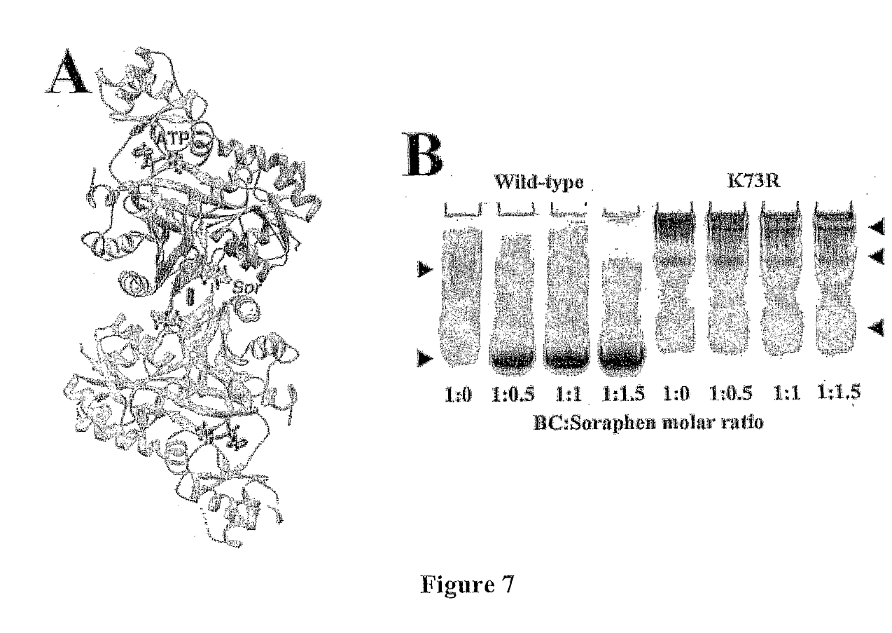

The BC subunits of bacterial ACCs are dimeric enzymes (FIG. 7A) (Thoden et al., supra (2000); Waldrop et al., supra (1994), and dimerization is essential for their activity (Janiyani, K. et al., J Biol Chem 276 (2001)). Similarly, yeast ACC is believed to function as a dimer or oligomer, while the isolated BC domain is monomeric in solution and is catalytically inactive (Weatherly et al., supra (2004)). The surface area of yeast BC domain that mediates the binding of soraphen A is equivalent to the dimer interface of the bacterial BC subunits (FIGS. 1C, 7A), and it is likely that the eukaryotic BC domains employ a similar mode of dimerization. Therefore, soraphen binding is expected to disrupt the dimerization of the BC domains, thereby leading to their inhibition. However, the exact molecular mechanism for the dimerization dependence of the activity of BC is currently not clear, as the two active sites of the BC dimer are located far from the dimer interface (FIG. 7A).

To obtain experimental evidence for the effects of soraphen A on the oligomerization state of BC domains, we examined the mobility of the yeast BC domain in a native gel electrophoresis assay. Similar observations were made using the BC domains of human ACC1 and Ustilago maydis ACC (data not shown) (Weatherly et al., supra (2004)). In the absence of soraphen A, wild-type BC domain runs as several smeared bands on the gel, suggesting various states of oligomerization (FIG. 7B). In the presence of soraphen A, a sharp band is observed, with the fastest migrating speed (FIG. 7B). Increasing the molar ratio between soraphen and the BC domain converts more of the protein into this fast migrating species (FIG. 7B). Based on our structural information, it is highly likely that this sharp band corresponds to the BC:soraphen complex, in a monomeric state, whereas the smeared bands with reduced mobility correspond to dimeric or oligomeric states of the enzyme (FIG. 7B). As a control, the mobility of the K73R mutant of the BC domain, which has drastically reduced affinity for soraphen (Table 3), is not affected by the presence of soraphen A (FIG. 7B). At the same time, the affinity for self-association of the isolated BC domain is likely to be low, as we observed only monomers in gel filtration and solution light scattering experiments (data not shown).

ACCs are attractive targets for the development of new therapeutic agents against obesity, diabetes and many other serious diseases. The eukaryotic ACCs possess two catalytic activities, embodied in the BC and the CT domains (FIG. 1A). Potent, small molecule inhibitors have been successfully identified and developed against the CT domain of this enzyme. For example, two classes of compounds have been used commercially as herbicides for more than 30 years (Delye, C. et al., Plant Physiol 132, 1716-1723 (2003); Devine, M. D., and Shukla, A. Crop Protection 19, 881-889 (2000); Gronwald, J. W. et al., Weed Science 39, 435-449 (1991); Zagnitko, O. et al., Proc Natl Acad Sci USA 98, 6617-6622 (2001)), both of which inhibit the CT domains of the ACC enzyme from sensitive plants (Rendina, A. R. et al., Arch Biochem Biophys 265, 219-225 (1988); Zhang et al., supra (2004)). More recently, potent inhibitors of mammalian ACCs have been identified by high-throughput screening, and kinetic and structural studies confirm that these compounds also function at the active site of the CT domain (Harwood Jr. et al., supra (2003); Zhang et al., supra (2004)). Up until now, soraphen is the only known potent inhibitor of the BC domain of eukaryotic ACCs. Its potent fungicidal activity demonstrates that inhibitors against the BC domain could also prove efficacious in the treatment of diseases linked to ACCs, opening a new avenue of discovery in the identification of inhibitors against these enzymes.

Polyketide natural products have become highly successful antibiotics, antivirals, anti-tumor agents, and immunosuppressants (Cane, D. E., Chem Rev 97, 2463-2464 (1997); Cane, D. E. et al., Science 282, -63-68 (1998); Walsh, C. T., Science 303, 1805-1810 (2004)). Our structural and biochemical studies reveal the novel molecular mechanism for the potent inhibitory activity of the polyketide soraphen A. The compound binds in the dimer interface and is a potent inhibitor of protein-protein interactions. The structural information should help the design and development of new soraphen analogs, with improved pharmacokinetic properties and reduced toxicity profiles, which may enable this natural product to become a broad-spectrum fungicide. The potent activity of this compound against human ACCs suggests the intriguing possibility that this natural product could also lead to compounds that are efficacious against obesity and diabetes.

TABLE 2
Summary of crystallographic information

| | BC: Soraphen A | BC |
|---|---|---|
| Structure | complex | Free enzyme |
| Resolution range (Å) | 30-1.8 | 30-2.5 |
| Number of observations | 564,576 | 110,250 |
| R_{merge}¹ (%) | 6.9 (32.5) | 8.0 (25.1) |
| I/σ | 19.1 (3.0) | 16.4 (3.6) |
| Observation redundancy | 3.6 (3.0) | 3.7 (3.4) |
| Number of reflections | 150,099 | 24,971 |
| Completeness (%) | 96 (88) | 84 (57) |
| R factor² (%) | 19.5 (25.8) | 25.4 (25.9) |
| Free R factor² (%) | 23.0 (28.3) | 32.3 (29.7) |
| rms deviation in bond lengths (Å) | 0.005 | 0.007 |
| rms deviation in bond angles (°) | 1.2 | 1.3 |

$1. {R}_{merge} = {\sum }_{h} {\sum }_{i} \vert {I}_{hi} - 〈 {I}_{h} 〉 \vert / {\sum }_{h} {\sum }_{i} {I}_{hi} .$
The numbers in parentheses are for the highest resolution shell.
$2. R = {\sum }_{h} \vert {F}_{h}^{o} - {F}_{h}^{c} \vert / {\sum }_{h} {F}_{h}^{o}$

TABLE 3
Affinity of soraphen for wild-type and mutant yeast BC domains.

| Yeast BC | | |
|---|---|---|
| domain | K_{d} (nM) (radiactive assay) | K_{d} (nM) (fluorescence assay) |
| Wild-type | 2.0 ± 0.9 | 3.9 ± 0.7 |
| I69E | —¹ | 104 ± 18 |
| K73R | —² | 2006 ± 174 |
| S77Y | —² | n. d.³ |
| E477R | 24.7 ± 10.4 | 274 ± 30 |
| N485G | 2.7 ± 0.4 | 55 ± 4 |
| W487R | —¹ | n. d.³ |
| F510I | 5.7 ± 1.0 | 10.6 ± 4.8 |

¹Detectable specific binding observed at up to 60 nM soraphen A (with 10 nM protein), but insifficient data for k_{d} determination.
²No specific binding observed at up to 60 nM soraphen A (with 10 nM protein)
³n. d.
—Not done.

Lengthy table referenced here
US20090215627A1-20090827-T00001
Please refer to the end of the specification for access instructions.

The foregoing is illustrative of the present invention, and is not to be construed as limiting thereof. The invention is defined by the following claims, with equivalents of the claims to be included therein.

LENGTHY TABLES
The patent application contains a lengthy table section. A copy of the table is available in electronic form from the USPTO web site (http://seqdata.uspto.gov/?pageRequest=docDetail&DocID=US20090215627A1). An electronic copy of the table will also be available from the USPTO upon request and payment of the fee set forth in 37 CFR 1.19(b)(3).

1. A crystal comprising a biotin carboxylase domain of eukaryotic acetyl-CoA carboxylase (ACC).

2. The crystal of claim 1, wherein said eukaryotic ACC is selected from the group consisting of yeast ACC, Ustilago ACC, Phytophthora ACC, Magnaporthe ACC, human ACC1 and human ACC2.

3. A computer-based method for identifying compounds that modulates activity of eukaryotic acetyl-CoA carboxylase comprising:
(a) providing at least 30 coordinates for a biotin carboxylase domain of acetyl-CoA carboxylase in a computer;
(b) providing a structure of a candidate compound to said computer in computer readable form; and
(c) determining whether or not said candidate compound fits into or docks with a binding cavity of said biotin carboxylase domain, wherein a candidate compound that fits or docks into said binding cavity is determined to be likely to modulate activity of eukaryotic acetyl-CoA carboxylase.

4. The method of claim 3 wherein said candidate compound is a member of a compound library.

5. A computer-based method for rationally designing a compound that modulates activity of eukaryotic acetyl-CoA carboxylase, comprising:
(a) generating a computer readable model of a binding site of a biotin carboxylase domain of eukaryotic acetyl-CoA carboxylase; and then
(b) designing in a computer with said model a compound having a structure and a charge distribution compatible with said binding site, said compound having a functional group that interacts with said binding site to modulate eukaryotic acetyl-CoA carboxylase activity.

6. A computer readable medium comprising the method of a claim 3.

7. A data structure comprising atomic coordinates for a biotin carboxylase domain of eukaryotic acetyl-CoA carboxylase.

8. A computer displaying a virtual model of a biotin carboxylase domain of eukaryotic acetyl-CoA carboxylase.

9. A storage medium containing atomic coordinates for a biotin carboxylase domain of eukaryotic acetyl-CoA carboxylase.

10. An organic compound produced by a method of claim 3, subject to the proviso that said compound is not soraphen A or an analog thereof.

11. The compound of claim 10, subject to the proviso that said compound is not a macrocyclic polyketide.

12. The compound of claim 10, wherein said compound (i) has a molecular weight of from 300 to 1000 Kilodaltons, (ii) includes a ring system, optionally substituted, of from 6 to 20 atoms, which ring system may optionally contain 1 to 5 hetero atoms selected from the group consisting of N, O and S, and (iii) which ring system has from 1 to 4 additional cyclic groups linked thereto.

13. The compound of claim 10, said compound selected from the group consisting of: 1,4-diazepine-2,5-diones, methyldecalins, piperazine-2,5-diones, and cytisines.

14. The compound of claim 10, which compound competitively inhibits the binding of soraphen A to a eukaryotic acetyl CoA carboxylase biotin carboxylase domain.

15. The compound of claim 14, wherein said acetyl CoA carboxylase biotin carboxylase domain is the biotin carboxylase domain of yeast ACC.

16. The compound of claim 10, which compound binds to a biotin carboxylase domain, and wherein the bound compound comes within seven angstroms of residues Lys73, Arg76, Ser77, Glu392, and Glu 477 of yeast ACC.

17. A method of treating a plant comprising administering a treatment-effective amount of a compound of claim 10 to said plant.

18. A method of treating metabolic syndrome in a subject in need of such treatment, comprising administering to said subject a compound of claim 10 in a treatment effective amount.

19. A method of treating insulin resistance syndrome in a subject in need of such treatment, comprising administering to said subject a compound of claim 10 in a treatment effective amount.

20. A method of treating obesity in a subject in need of such treatment, comprising administering to said subject a compound of claim 10 in a treatment effective amount.

21. A composition comprising a compound of claim 10 in an agriculturally acceptable carrier.

22. A pharmaceutical composition comprising a compound of claim 10 in a pharmaceutically acceptable carrier.

23. A computer readable medium comprising the method of claim 5.

24. An organic compound produced by a method of claim 5 subject to the proviso that said compound is not soraphen A or an analog thereof.


SEQUENCE LISTING (12 sequences)

SEQ ID NO: 1 - protein, 538 aa, Ustilago maydis
ASPVADFIRKQGGHSVITKVLICNNGIAAVKEIRSIRKWAYETFGDERAIEFTVMATPEDLKVNADYIRMADQYVEVPGGSNNNNYANVDLIVDVAERAGVHAVWAGWGHASENPRLPESLAASKHKIIFIGPPGSAMRSLGDKISSTIVAQHADVPCMPWSGTGIKETMMSDQGFLTVSDDVYQQACIHTAEEGLEKAEKIGYPVMIKASEGGGGKGIRKCTNGEEFKQLYNAVLGEVPGSPVFVMKLAGQARHLEVQLLADQYGNAISIFGRDCSVQRRHQKIIEEAPVTIAPEDARESMEKAAVRLAKLVGYVSAGTVEWLYSPESGEFAFLELNPRLQVEHPTTEMVSGVNIPAAQLQVAMGIPLYSIRDIRTLYGMDPRGNEVIDFDFSSPESFKTQRKPQPQGHVVACRITAENPDTGFKPGMGALTELNFRSSTSTWGYFSVGTSGALHEYADSQFGHIFAYGADRSEARKQMVISLKELSIRGDFRTTVEYLIKLLETDAFESNKITTGWLDGLIQDRLTAERPPADLAV

SEQ ID NO: 2 - protein, 531 aa, Homo sapiens
VASPAEFVTRFGGNKVIEKVLIANNGIAAVKCMRSIRRWSYEMFRNERAIRFVVMVTPEDLKANAEYIKMADHYVPVPGGPNNNNYANVELILDIAKRIPVQAVWAGWGHASENPKLPELLLKNGIAFMGPPSQAMWALGDKIASSIVAQTAGIPTLPWSGSGLRVDWQENDFSKRILNVPQELYEKGYVKDVDDGLQAAEEVGYPVMIKASEGGGGKGIRKVNNADDFPNLFRQVQAEVPGSPIFVMRLAKQSRHLEVQILADQYGNAISLFGRDCSVQRRHQKIIEEAPATIATPAVFEHMEQCAVKLAKMVGYVSAGTVEYLYSQDGSFYFLELNPRLQVEHPCTEMVADVNLPAAQLQIAMGIPLYRIKDIRMMYGVSPWGDSPIDFEDSAHVPCPRGHVIAARITSENPDEGFKPSSGTVQELNFRSNKNVWGYFSVAAAGGLHEFADSQFGHCFSWGENREEAISNMVVALKELSIRGDFRTTVEYLIKLLETESFQMNRIDTGWLDRLIAEKVQAERPDTMLGV

SEQ ID NO: 3 - protein, 532 aa, Homo sapiens
VASPAEFVTRFGGDRVIEKVLIANNGIAAVKCMRSIRRWAYEMFRNERAIRFVVMVTPEDLKANAEYIKMADHYVPVPGGPNNNNYANVELIVDIAKRIPVQAVWAGWGHASENPKLPELLCKNGVAFLGPPSEAMWALGDKIASTVVAQTLQVPTLPWSGSGLTVEWTEDDLQQGKRISVPEDVYDKGCVKDVDEGLEAAERIGFPLMIKASEGGGGKGIRKAESAEDFPILFRQVQSEIPGSPIFLMKLAQHARHLEVQILADQYGNAVSLFGRDCSIQRRHQKIVEEAPATIAPLAIFEFMEQCAIRLAKTVGYVSAGTVEYLYSQDGSFHFLELNPRLQVEHPCTEMIADVNLPAAQLQIAMGVPLHRLKDIRLLYGESPWGVTPISFETPSNPPLARGHVIAARITSENPDEGFKPSSGTVQELNFRSSKNVWGYFSVAATGGLHEFADSQFGHCFSWGENREEAISNMVVALKELSIRGDFRTTVEYLINLLETESFQNNDIDTGWLDYLIAEKVQAEKPDIMLGV

SEQ ID NO: 4 - protein, 559 aa, Ustilago maydis
PPPDHKAVSQFIGGNPLETAPASPVADFIRKQGGHSVITKVLICNNGIAAVKEIRSIRKWAYETFGDERAIEFTVMATPEDLKVNADYIRMADQYVEVPGGSNNNNYANVDLIVDVAERAGVHAVWAGWGHASENPRLPESLAASKHKIIFIGPPGSAMRSLGDKISSTIVAQHADVPCMPWSGTGIKETMMSDQGFLTVSDDVYQQACIHTAEEGLEKAEKIGYPVMIKASEGGGGKGIRKCTNGEEFKQLYNAVLGEVPGSPVFVMKLAGQARHLEVQLLADQYGNAISIFGRDCSVQRRHQKIIEEAPVTIAPEDARESMEKAAVRLAKLVGYVSAGTVEWLYSPESGEFAFLELNPRLQVEHPTTEMVSGVNIPAAQLQVAMGIPLYSIRDIRTLYGMDPRGNEVIDFDFSSPESFKTQRKPQPQGHVVACRITAENPDTGFKPGMGALTELNFRSSTSTWGYFSVGTSGALHEYADSQFGHIFAYGADRSEARKQMVISLKELSIRGDFRTTVEYLIKLLETDAFESNKITTGWLDGLIQDRLTAERPPADLAV

SEQ ID NO: 5 - protein, 632 aa, Homo sapiens
MDEPSPLAQPLELNQHSRFIIGSVSEDNSEDEISNLVKLDLLEEKEGSLSPASVGSDTLSDLGISSLQDGLALHIRSSMSGLHLVKQGRDRKKIDSQRDFTVASPAEFVTRFGGNKVIEKVLIANNGIAAVKCMRSIRRWSYEMFRNERAIRFVVMVTPEDLKANAEYIKMADHYVPVPGGPNNNNYANVELILDIAKRIPVQAVWAGWGHASENPKLPELLLKNGIAFMGPPSQAMWALGDKIASSIVAQTAGIPTLPWSGSGLRVDWQENDFSKRILNVPQELYEKGYVKDVDDGLQAAEEVGYPVMIKASEGGGGKGIRKVNNADDFPNLFRQVQAEVPGSPIFVMRLAKQSRHLEVQILADQYGNAISLFGRDCSVQRRHQKIIEEAPATIATPAVFEHMEQCAVKLAKMVGYVSAGTVEYLYSQDGSFYFLELNPRLQVEHPCTEMVADVNLPAAQLQIAMGIPLYRIKDIRMMYGVSPWGDSPIDFEDSAHVPCPRGHVIAARITSENPDEGFKPSSGTVQELNFRSNKNVWGYFSVAAAGGLHEFADSQFGHCFSWGENREEAISNMVVALKELSIRGDFRTTVEYLIKLLETESFQMNRIDTGWLDRLIAEKVQAERPDTMLGV

SEQ ID NO: 6 - protein, 775 aa, Homo sapiens
MVLLLCLSCLIFSCLTFSWLKIWGKMTDSKPITKSKSEANLIPSQEPFPASDNSGETPQRNGEGHTLPKTPSQAEPASHKGPKDAGRRRNSLPPSHQKPPRNPLSSSDAAPSPELQANGTGTQGLEATDTNGLSSSARPQGQQAGSPSKEDKKQANIKRQLMTNFILGSFDDYSSDEDSVAGSSRESTRKGSRASLGALSLEAYLTTGEAETRVPTMRPSMSGLHLVKRGREHKKLDLHRDFTVASPAEFVTRFGGDRVIEKVLIANNGIAAVKCMRSIRRWAYEMFRNERAIRFVVMVTPEDLKANAEYIKMADHYVPVPGGPNNNNYANVELIVDIAKRIPVQAVWAGWGHASENPKLPELLCKNGVAFLGPPSEAMWALGDKIASTVVAQTLQVPTLPWSGSGLTVEWTEDDLQQGKRISVPEDVYDKGCVKDVDEGLEAAERIGFPLMIKASEGGGGKGIRKAESAEDFPILFRQVQSEIPGSPIFLMKLAQHARHLEVQILADQYGNAVSLFGRDCSIQRRHQKIVEEAPATIAPLAIFEFMEQCAIRLAKTVGYVSAGTVEYLYSQDGSFHFLELNPRLQVEHPCTEMIADVNLPAAQLQIAMGVPLHRLKDIRLLYGESPWGVTPISFETPSNPPLARGHVIAARITSENPDEGFKPSSGTVQELNFRSSKNVWGYFSVAATGGLHEFADSQFGHCFSWGENREEAISNMVVALKELSIRGDFRTTVEYLINLLETESFQNNDIDTGWLDYLIAEKVQAEKPDIMLGV

SEQ ID NO: 7 - protein, 554 aa, Phytophthora infestans
VAEEAPPAADVAAYAETRSDSNPLNYASMEEYVRLQKGTRPITSVLIANNGISAVKAIRSIRSWSYEMFADEHVVTFVVMATPEDLKANAEYIRMAEHVVEVPGGSNNHNYANVSLIIEIAERFNVDAVWAGWGHASENPLLPDTLAQTERKIVFIGPPGKPMRALGDKIGSTIIAQSAKVPTIAWNGDGMEVDYKEHDGIPDEIYNAAMLRDGQHCLDECKRIGFPVMIKASEGGGGKGIRMVHEESQVLSAWEAVRGEIPGSPIFVMKLAPKSRHLEVQLLADTYGNAIALSGRDCSVQRRHQKIVEEGPVLAPTQEVWEKMMRAATRLAQEVEYVNAGTVEYLFSELPEDNGNSFFFLELNPRLQVEHPVTEMITHVNLPAAQLQVAMGIPLHCIPDVRRLYNKDAFETTVIDFDAEKQKPPHGHVIAARITAEDPNAGFQPTSGAIQELNFRSTPDVWGYFSVDSSGQVHEFADSQIGHLFSWSPTREKARKNMVLALKELSIRGDIHTTVEYIVNMMESDDFKYNRISTSWLDERISHHNEVRLQGRPD

SEQ ID NO: 8 - protein, 580 aa, Saccharomyces cerevisiae
SEESLFESSPQKMEYEITNYSERHTELPGHFIGLNTVDKLEESPLRDFVKSHGGHTVISKILIANNGIAAVKEIRSVRKWAYETFGDDRTVQFVAMATPEDLEANAEYIRMADQYIEVPGGTNNNNYANVDLIVDIAERADVDAVWAGWGHASENPLLPEKLSQSKRKVIFIGPPGNAMRSLGDKISSTIVAQSAKVPCIPWSGTGVDTVHVDEKTGLVSVDDDIYQKGCCTSPEDGLQKAKRIGFPVMIKASEGGGGKGIRQVEREEDFIALYHQAANEIPGSPIFIMKLAGRARHLEVQLLADQYGTNISLFGRDCSVQRRHQKIIEEAPVTIAKAETFHEMEKAAVRLGKLVGYVSAGTVEYLYSHDDGKFYFLELNPRLQVEHPTTEMVSGVNLPAAQLQIAMGIPMHRISDIRTLYGMNPHSASEIDFEFKTQDATKKQRRPIPKGHCTACRITSEDPNDGFKPSGGTLHELNFRSSSNVWGYFSVGNNGNIHSFSDSQFGHIFAFGENRQASRKHMVVALKELSIRGDFRTTVEYLIKLLETEDFEDNTITTGWLDDLITHKMTAEKPDPTLAV

SEQ ID NO: 9 - protein, 591 aa, Magnaporthe grisea
TETNGTAAAANSSRQRNGANGVTVPVANGKATYAQRHKIADHFIGGNRLENAPPSKVKEWVAAHDGHTVITNVLIANNGIAAVKEIRSVRKWAYETFGDERAIQFTVMATPEDLQANADYIRMADHYVEVPGGTNNNNYANVELIVDVAERMNVHAVWAGWGHASENPKLPESLAASPKKIIFIGPPGSAMRSLGDKISSTIVAQHAQVPCIPWSGTGVDAVQIDKKGIVTVDDDTYAKGCVTSWQEGLEKARQIGFPVMIKASEGGGGKGIRKAVSEEGFEELYKAAASEIPGSPIFIMKLAGNARHLEVQLLADQYGNNISLFGRDCSVQRRHQKIIEEAPVTIAKPDTFKAMEEAAVRLGRLVGYVSAGTVEYLYSHADDKFYFLELNPRLQVEHPTTEGVSGVNLPASQLQIAMGIPLHRISDIRLLYGVDPKLSTEIDFDFKNPDSEKTQRRPSPKGHLTACRITSEDPGEGFKPSNGVMHELNFRSSSNVWGYFSVGTQGGIHSFSDSQFGHIFAYGENRSASRKHMVIALKELSIRGDFRTTVEYLIKLLETEAFEENTITTGWLDELISKKLTAERPDKMLAV

SEQ ID NO: 10 - protein, 581 aa, Saccharomyces cerevisiae
MSEESLFESSPQKMEYEITNYSERHTELPGHFIGLNTVDKLEESPLRDFVKSHGGHTVISKILIANNGIAAVKEIRSVRKWAYETFGDDRTVQFVAMATPEDLEANAEYIRMADQYIEVPGGTNNNNYANVDLIVDIAERADVDAVWAGWGHASENPLLPEKLSQSKRKVIFIGPPGNAMRSLGDKISSTIVAQSAKVPCIPWSGTGVDTVHVDEKTGLVSVDDDIYQKGCCTSPEDGLQKAKRIGFPVMIKASEGGGGKGIRQVEREEDFIALYHQAANEIPGSPIFIMKLAGRARHLEVQLLADQYGTNISLFGRDCSVQRRHQKIIEEAPVTIAKAETFHEMEKAAVRLGKLVGYVSAGTVEYLYSHDDGKFYFLELNPRLQVEHPTTEMVSGVNLPAAQLQIAMGIPMHRISDIRTLYGMNPHSASEIDFEFKTQDATKKQRRPIPKGHCTACRITSEDPNDGFKPSGGTLHELNFRSSSNVWGYFSVGNNGNIHSFSDSQFGHIFAFGENRQASRKHMVVALKELSIRGDFRTTVEYLIKLLETEDFEDNTITTGWLDDLITHKMTAEKPDPTLAV

SEQ ID NO: 11 - protein, 570 aa, Homo sapiens
GISSLQDGLALHIRSSMSGLHLVKQGRDRKKIDSQRDFTVASPAEFVTRFGGNKVIEKVLIANNGIAAVKCMRSIRRWSYEMFRNERAIRFVVMVTPEDLKANAEYIKMADHYVPVPGGPNNNNYANVELILDIAKRIPVQAVWAGWGHASENPKLPELLLKNGIAFMGPPSQAMWALGDKIASSIVAQTAGIPTLPWSGSGLRVDWQENDFSKRILNVPQELYEKGYVKDVDDGLQAAEEVGYPVMIKASEGGGGKGIRKVNNADDFPNLFRQVQAEVPGSPIFVMRLAKQSRHLEVQILADQYGNAISLFGRDCSVQRRHQKIIEEAPATIATPAVFEHMEQCAVKLAKMVGYVSAGTVEYLYSQDGSFYFLELNPRLQVEHPCTEMVADVNLPAAQLQIAMGIPLYRIKDIRMMYGVSPWGDSPIDFEDSAHVPCPRGHVIAARITSENPDEGFKPSSGTVQELNFRSNKNVWGYFSVAAAGGLHEFADSQFGHCFSWGENREEAISNMVVALKELSIRGDFRTTVEYLIKLLETESFQMNRIDTGWLDRLIAEKVQAERPDTMLGV

SEQ ID NO: 12 - protein, 449 aa, Escherichia coli
MLDKIVIANRGEIALRILRACKELGIKTVAVHSSADRDLKHVLLADETVCIGPAPSVKSYLNIPAIISAAEITGAVAIHPGYGFLSENANFAEQVERSGFIFIGPKAETIRLMGDKVSAIAAMKKAGVPCVPGSDGPLGDDMDKNRAIAKRIGYPVIIKASGGGGGRGMRVVRGDAELAQSISMTRAEAKAAFSNDMVYMEKYLENPRHVEIQVLADGQGNAIYLAERDCSMQRRHQKVVEEAPAPGITPELRRYIGERCAKACVDIGYRGAGTFEFLFENGEFYFIEMNTRIQVEHPVTEMITGVDLIKEQLRIAAGQPLSIKQEEVHVPGHAVECRINAEDPNTFLPSPGKITRFHAPGGFGVRWESHIYAGYTVPPYYDSMIGKLICYGENRDVAIARMKNALQELIIDGIKTNVDLQIRIMNDENFQHGGTNIHYLEKKLGLQEK